(19) United States
(12) Patent Application Publication
(10) Pub. No.: US 2023/0383357 A1
(43) Pub. Date: Nov. 30, 2023

(54) SUBJECT-SPECIFIC TREATMENTS FOR VENETOCLAX-RESISTANT ACUTE MYELOID LEUKEMIA

(71) Applicant: The Regents of the University of Colorado, A Body Corporate, Denver, CO (US)

(72) Inventors: Craig T. JORDAN, Aurora, CO (US); Clayton SMITH, Denver, CO (US); Shanshan PEI, Yuhang, Hangzhou (CN); Brett STEVENS, Aurora, CO (US); Austin E. GILLEN, Kittredge, CO (US)

(21) Appl. No.: 18/030,627

(22) PCT Filed: Oct. 6, 2021
(86) PCT No.: PCT/US2021/053709

§ 371 Date: Apr. 6, 2023

Related U.S. Application Data
(60) Provisional application No. 63/087,998, filed on Oct. 6, 2020.

(51) Int. Cl. C12Q 1/6886

(52) U.S. Cl. CPC C12Q 1/6886; C12Q 2600/106; C12Q 2600/158

(57) ABSTRACT

The present disclosure provides methods of treating venetoclax-resistant acute myeloid leukemia, including methods of identifying alternative treatment targets in specific subsets of patients who would otherwise be resistant to treatment with venetoclax and/or azacitidine.

### RELATED APPLICATIONS

This application claims priority to, and benefit of U.S. Provisional Application No. 63/087,998, filed Oct. 6, 2020, the contents of which are incorporated herein by reference in its entirety.

### TECHNICAL FIELD

The invention relates to improvements in subject-specific treatments for venetoclax-resistant acute myeloid leukemia.

### BACKGROUND OF THE INVENTION

Acute myeloid leukemia (AML) is a blood cancer in which the bone marrow of a subject makes abnormal myeloblasts, red blood cells, or platelets. AML is one of the most common forms of acute leukemia in adults. The build-up of AML cells in bone marrow and blood can rapidly lead to infection, anemia, excessive bleeding and death. BCL-2 inhibitor venetoclax has recently emerged as an important component of therapy for acute myeloid leukemia (AML). The current FDA-approved standard of care for the majority of patients who are too elderly or unfit for aggressive chemotherapy is treatment with venetoclax in combination with a hypomethylating agent, such as azacitidine (“Ven/aza treatment”) or decitabine. It is estimated that approximately 70% of these patients will achieve complete remission (CR) of their disease upon Ven/aza treatment. However, it is estimated that approximately 30% of patients do not respond to treatment with ven/aza and are unable to achieve CR. There is an unmet need in the art for methods of identifying subjects that will not respond to treatment with Ven/aza, as well as for methods of treating such subjects. The present disclosure addresses this need.

### SUMMARY OF THE INVENTION

The present disclosure provides a method of identifying a subject having acute myeloid leukemia (AML) that will be resistant to treatment with a combination of venetoclax and azacitidine, the method comprising: a) measuring the expression levels of at least 10 genes in a plurality of leukemia cells isolated from a biological sample from the subject, wherein the at least 10 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) identifying that the subject will be resistant to treatment with a combination of venetoclax and azacitidine when the percentage from step (c) is greater than the predetermined cutoff percentage.

The present disclosure provides a method of identifying a subject having acute myeloid leukemia (AML) that will be resistant to treatment with a combination of venetoclax and azacitidine, the method comprising: a) measuring the expression levels of at least 10 genes in a plurality of leukemia cells isolated from a biological sample from the subject, wherein the at least 10 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) identifying that the subject will be resistant to treatment with a combination of venetoclax and azacitidine when the percentage from step (c) is greater than the predetermined cutoff percentage.

In some aspects, classifying a measured cell as resistant to treatment with a combination of venetoclax and azacitidine or classifying a measured cell as responsive to treatment with a combination of venetoclax and azacitidine based on the expression levels measured in step (a) can comprise: i) determining a score based on the expression level of the at least 10 genes, wherein the score is determined using a machine learning classifier; ii) comparing the score determined in step (i) to a predetermined cutoff value; and iii) classifying the cell as resistant to treatment with a combination of venetoclax and azacitidine when the score is greater than or equal to the predetermined cutoff value or classifying the cell as responsive to treatment with a combination of venetoclax and azacitidine when the score is less than the predetermined cutoff value.

In some aspects, classifying a measured cell as resistant to treatment with a combination of venetoclax and azacitidine or classifying a measured cell as responsive to treatment with a combination of venetoclax and azacitidine based on the expression levels measured in step (a) can comprise: i) determining a score based on the expression level of the at least 10 genes, wherein the score is determined using a machine learning classifier; ii) comparing the score determined in step (i) to a predetermined cutoff value; and iii) classifying the cell as resistant to treatment with a combination of venetoclax and azacitidine when the score is less than or equal to the predetermined cutoff value or classifying the cell as responsive to treatment with a combination of venetoclax and azacitidine when the score is greater than the predetermined cutoff value.

In some aspects, a machine learning classifier can be trained and validated using the expression levels of the at least 10 genes measured in at least two training samples, wherein at least one of the at least two training samples comprises leukemia cells isolated from a subject that is responsive to treatment with venetoclax and azacitidine, and wherein at least one of the at least two training samples comprises leukemia cells isolated from a subject that is resistant to treatment with venetoclax and azacitidine.

In some aspects of the preceding methods, step (a) can comprise measuring the expression levels of at least 25 genes in the plurality of leukemia cells, wherein the at least 25 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7.

In some aspects of the preceding methods, step (a) can comprise measuring the expression levels of at least 25 genes in the plurality of leukemia cells, wherein the at least 25 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, G0S2, GNLY, HBD, HPGD, IF127, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C.

In some aspects of the preceding methods, step (a) can comprise measuring the expression levels of at least 40 genes in the plurality of leukemia cells, wherein the at least 40 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7.

In some aspects of the preceding methods, step (a) can comprise measuring the expression levels of at least 40 genes in the plurality of leukemia cells, wherein the at least 40 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, G0S2, GNLY, HBD, HPGD, IF127, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C.

In some aspects, a plurality of leukemia cells can comprise at least about 300 leukemia cells. Leukemia cells can comprise acute myeloid leukemia cells. Leukemia cells can comprise acute myeloid leukemia blast cells. Leukemia cells can comprise leukemia stem cells. Leukemia stem cells can comprise reactive oxygen species-low leukemia stem cells.

In some aspects, a predetermined cutoff percentage can be at least about 25%.

The preceding methods can further comprise providing a treatment recommendation to the subject that is identified as a subject that is resistant to treatment with a combination of venetoclax and azacitidine, wherein the treatment recommendation comprises recommending the administration of at least one therapeutically effective amount of at least one alternative therapy. The preceding methods can further comprise administering to the subject identified as resistant to treatment with a combination of venetoclax and azacitidine at least one therapeutically effective amount of at least one alternative therapy. An least one alternative therapy can comprise anti-cancer therapy, chemotherapy, targeted drug therapy, radiation therapy, immunotherapy, stem cell transplant or any combination thereof.

The present disclosure provides a method of providing an AML treatment recommendation for a subject, the method comprising: a) determining the expression level of at least one gene in a plurality of leukemia stem cells isolated from a biological sample from the subject, wherein the at least one gene is selected from NFκB, mTOR, RSK, ERK, MEK, stat3, src, mcl1; b) comparing the expression level of the at least one gene in the measured cells to a corresponding predetermined cutoff value; c) determining the percentage of leukemia cells in the plurality of leukemia cells that exhibit an expression level of the at least one gene that is greater than the corresponding predetermined cutoff value; d) comparing the percentage from step (c) to a predetermined cutoff percentage; and e) recommending a treatment comprising the administration of at least one therapeutically effective amount of at least one agent that targets the PI3K/AKT/mTOR pathway when the percentage from step (c) is greater than the predetermined cutoff percentage.

In some aspects, an at least one agent that targets the PI3K/AKT/mTOR can be an agent that inhibits at least one of PI3K, AKT and mTOR. An at least one agent that targets the PI3K/AKT/mTOR pathway can be selected from everolimus, temsirolimus, sirolimus, CC-223, vistusertib, nab-rapamycin, CC-115, sapanisertib, copanlisib, duvelisib, alpelisib, idelalisib, puquitinib, leniolisib, buparlisib, RTB 101, umbralisib, TG-100-115, nemiralisib, GSK2636771, fimepinostat, tenalisib, serabelisib, INCB50465, SF1126, GDC-0077, AZD8186, ME401, IPI-549, MEN 1611, ASN003, bimiralisib, GDC0084, voxtalisib, LY3023414, gedatolisib, ARG-092, MK-2206, iapatasertib, uprosertib, capivasertib, triciribine, ARQ-751, PF-04979064 and PF-04691502.

The present disclosure provides a method of providing an AML treatment recommendation for a subject, the method comprising: a) determining the expression level of at least one gene in a plurality of leukemia stem cells isolated from a biological sample from the subject, wherein the at least one gene is selected from CD38, LAMPS, SLC44A1 (CD92), PLAC8, NCAM1 (CD56) and CD70; b) comparing the expression level of the at least one gene in the measured cells to a corresponding predetermined cutoff value; c) determining the percentage of leukemia cells in the plurality of leukemia cells that exhibit an expression level of the at least one gene that is greater than the corresponding predetermined cutoff value; d) comparing the percentage from step (c) to a predetermined cutoff percentage; e) recommending a treatment comprising the administration of at least one therapeutically effective amount of at least one agent that targets the at least one gene when the percentage from step (c) is greater than the predetermined cutoff percentage.

In some aspects of the preceding method, the at least one gene can be CD38. In some aspects of the preceding method, the at least one gene can CD38 and the at least one agent can be daratumumab.

In some aspects of the preceding methods, the at least one gene can be LAMPS. In some aspects of the preceding methods, the at least one gene can be LAMPS and the at least one agent can be pinometostat.

In some aspects of the preceding methods, the at least one gene can be PLAC8. In some aspects of the preceding methods, the at least one gene can be PLAC8 and the at least one agent can be a PI3K inhibitor. The PI3K inhibitor can be selected from copanlisib, duvelisib, alpelisib, idelalisib, puquitinib, leniolisib, buparlisib, RTB101, umbralisib, TG-100-115, nemiralisib, GSK2636771, fimepinostat, tenalisib, serabelisib, INCB50465, SF1126, GDC-0077, AZD8186, ME401, IPI-549, MEN 1611 and ASN003.

In some aspects of the preceding methods, the at least one gene can be NCAM1 (CD56). In some aspects of the preceding method, the at least one gene can be NCAM1 (CD56) and the at least one agent can be lorvotuzumab or mertansine.

In some aspects of the preceding methods, the at least one gene can be SLC44A1 (CD92).

In some aspects of the preceding methods, the at least one gene can be CD70.

In some aspects of the preceding methods, the at least one agent can be an antibody or a CAR-T cell.

In some aspects of the preceding methods, a treatment can further comprise the administration of at least one therapeutically effective amount of venetoclax, azacitidine or a combination of venetoclax and azacitidine.

In some aspects of the preceding methods, determining the expression level can comprise PCR, high-throughput sequencing, next generation sequencing, RNA-sequencing, Northern Blot, reverse transcription PCR (RT-PCR), real-time PCR (qPCR), quantitative PCR, qRT-PCR, flow cytometry, mass spectrometry, microarray analysis, digital droplet PCR, Western Blot or any combination thereof. RNA-sequencing can be Cellular Indexing of Transcriptomes and Epitopes by Sequencing (CITE-Seq).

In some aspects of the preceding methods, a biological sample can comprise blood, a bone marrow biopsy, a bone marrow aspirate, a biopsy of a chloroma, a tissue biopsy, cerebrospinal fluid or any combination thereof.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” Unless specifically stated or obvious from context, as used herein, the term “or” is understood to be inclusive and covers both “or” and “and”.

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is a bar chart showing the number of individual cells within the Ven/aza^{R} AML, validation samples and Ven/aza^{S} AML validation samples classified as being resistant to Ven/aza treatment (R) or classified as being sensitive to Ven/aza treatment (S) using the methods of the present disclosure.

FIG. 2 is a chart showing the percentage of individual cells in each of the Ven/aza^{R} AML, validation samples and Ven/aza^{S} AML validation samples classified as being resistant to Ven/aza treatment using the methods of the present disclosure.

FIG. 3 shows a heat map of the phosphoflow staining intensity for a variety of different proteins in untreated and Ven/aza treated samples, both from patients who responded to Ven/aza treatment, and patients who were resistant to Ven/aza treatment.

FIG. 4 is a graph showing the cell viability in AML samples treated with either venetoclax (VEN), a combination of venetoclax and azacitidine (VEN/AZA), the dual PI3K/mTOR inhibitor PF-04979064 (PZ), a combination of PF-04979064 and venetoclax (PZ/VEN), and a combination of PF-04979064, venetoclax and azacitidine (PZ/VEN/AZA)

FIG. 5 is a bar chart showing the number of individual cells within the Ven/aza^{R} AML validation samples and Ven/aza^{S} AML validation samples classified as being resistant to Ven/aza treatment (R) or classified as being sensitive to Ven/aza treatment (S) using the methods of the present disclosure.

FIG. 6 is a chart showing the percentage of individual cells in each of the Ven/aza^{R} AML validation samples and Ven/aza^{S} AML validation samples classified as being resistant to Ven/aza treatment using the methods of the present disclosure.

FIG. 7 is a bar chart showing the number of individual cells within three diagnosis/relapse pairs of Ven/aza^{S} AML samples classified as being resistant to Ven/aza treatment (R) or classified as being sensitive to Ven/aza treatment (S) using the methods of the present disclosure.

FIG. 8 is a chart showing the percentage of individual cells in each of the paired diagnosis and relapse AML samples classified as being resistant to Ven/aza treatment using the methods of the present disclosure. Lines connect the paired diagnosis and relapse samples.

### DETAILED DESCRIPTION OF THE INVENTION

Acute myeloid leukemia is a blood cancer that is one of the most commonly diagnosed types of leukemia in adults. It is estimated that there will be approximately 11,000 deaths from AML in the United States in 2020, along with 20,000 newly diagnosed cases. The average age of a person diagnosed with acute myeloid leukemia is about 68, with most cases occurring after the age of 45. However, acute myeloid leukemia has also been diagnosed in younger patients, including children. Prognosis for patients diagnosed with acute myeloid leukemia is generally poor, with a long-term survival of only 40-50% in younger patients and a median overall survival of less than one year for older patients. New therapies aimed at supplementing the standard remission induction regimen of infusional cytarabine with intermittent dosing of an anthracycline have not yielded additional clinical benefits. Thus, there exists a need for more specialized and personalized treatment methods, particularly in older patients who are unfit for induction therapy.

Recent research has demonstrated that acute myeloid leukemia exhibits a high level of biological heterogeneity, potentially explaining the difficulty in finding effective therapeutic strategies for the treatment of AML. Furthermore, it has been recently recognized that leukemia stem cells (LSCs), which are capable of giving rise to identical daughter cells as well as differentiated cells, perpetuate and maintain acute myeloid leukemia.

As an alternative to standard induction therapy in, the current FDA-approved standard of care for elderly patients or patients who are otherwise unfit for such an aggressive chemotherapy is treatment with a combination of the BCL-2 inhibitor venetoclax and a hypomethylating agent (HMA), such as azacitidine or decitabine. Specifically, treatment with a combination of venetoclax and azacitidine (hereafter referred to as “Ven/aza treatment” or “treatment with Ven/aza”) is estimated to induce a complete remission (CR) of AML in approximately 70% of treated patients.

However, this means that approximately 30% of patients do not end up responding to Ven/aza treatment and therefore do not achieve complete remission. There is a need in the art for methods of identifying this 30% of patients who are unlikely to respond to treatment with Ven/aza. The ability to identify these patients, prior to treatment, would allow clinicians to avoid the toxicity, expense and negative quality of life associated with an ineffective therapy. Moreover, these patients could be directed to other therapies, increasing their odds of survival. Finally, having a reliable method of identifying these patients will allow for the design of clinical trials aimed at testing personalized therapies for this specific AML patient population.

The present disclosure provides methods for the treatment and prognosis of acute myeloid leukemia in a subject. Various methods of the present disclosure are described in full detail herein.

The present disclosure provides a method of identifying a subject having acute myeloid leukemia (AML) that will be resistant to treatment with a combination of venetoclax and azacitidine, the method comprising: a) measuring the expression levels of at least 2 genes in a plurality of leukemia cells isolated from a biological sample from the subject, wherein the at least 2 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) identifying that the subject will be resistant to treatment with a combination of venetoclax and azacitidine when the percentage from step (c) is greater than the predetermined cutoff percentage.

The present disclosure provides a method of identifying a subject having acute myeloid leukemia (AML) that will be resistant to treatment with a combination of venetoclax and azacitidine, the method comprising: a) measuring the expression levels of at least 2 genes in a plurality of leukemia cells isolated from a biological sample from the subject, wherein the at least 2 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) identifying that the subject will be resistant to treatment with a combination of venetoclax and azacitidine when the percentage from step (c) is greater than the predetermined cutoff percentage.

In some aspects, the preceding methods can further comprise providing a treatment recommendation to a subject that is identified as a subject that is resistant to treatment with a combination of venetoclax and azacitidine. In some aspects, the treatment recommendation can comprise recommending the administration of at least one therapeutically effective amount of at least one alternative therapy.

In some aspects, the preceding methods can further comprise administering to the subject identified as resistant to treatment with a combination of venetoclax and azacitidine at least one therapeutically effective amount of at least one alternative therapy.

The present disclosure provides a method of treating AML in a subject, the method comprising: a) measuring the expression levels of at least 2 genes in a plurality of leukemia cells isolated from a biological sample from the subject, wherein the at least 2 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) administering to the subject at least one therapeutically effective amount of at least one alternative therapy when the percentage from step (c) is greater than the predetermined cutoff percentage.

The present disclosure provides a method of treating AML in a subject, the method comprising: a) measuring the expression levels of at least 2 genes in a plurality of leukemia cells isolated from a biological sample from the subject, wherein the at least 2 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) administering to the subject at least one therapeutically effective amount of at least one alternative therapy when the percentage from step (c) is greater than the predetermined cutoff percentage.

In some aspects of the preceding methods, step (a) can comprise measuring the expression levels of at least 3 genes, or at least 4 genes, or at least 5 genes, or at least 6 genes, or at least 7 genes or at least 8 genes, or at least 9 genes, or at least 10 genes, or at least 11 genes, or at least 12 genes, or at least 13 genes, or at least 14 genes, or at least 15 genes, or at least 16 genes, or at least 17 genes, or at least 18 genes, or at least 19 genes, or at least 20 genes, or at least 21 genes, or at least 22 genes, or at least 23 genes, or at least 24 genes, or at least 25 genes, or at least 26 genes, or at least 27 genes, or at least 28 genes, or at least 29 genes, or at least 30 genes, or at least 31 genes, or at least 32 genes, or at least 33 genes, or at least 34 genes, or at least 35 genes, or at least 36 genes, or at least 37 genes, or at least 38 genes, or at least 39 genes, or at least 40 genes, or at least 41 genes, or at least 42 genes, or at least 43 genes, or at least 44 genes, or at least 45 genes, or at least 46 genes, or at least 47 genes, or at least 48 genes, or at least 49 genes, or at least 50 genes selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7.

In some aspects of the preceding methods, step (a) can comprise measuring the expression levels of at least 3 genes, or at least 4 genes, or at least 5 genes, or at least 6 genes, or at least 7 genes or at least 8 genes, or at least 9 genes, or at least 10 genes, or at least 11 genes, or at least 12 genes, or at least 13 genes, or at least 14 genes, or at least 15 genes, or at least 16 genes, or at least 17 genes, or at least 18 genes, or at least 19 genes, or at least 20 genes, or at least 21 genes, or at least 22 genes, or at least 23 genes, or at least 24 genes, or at least 25 genes, or at least 26 genes, or at least 27 genes, or at least 28 genes, or at least 29 genes, or at least 30 genes, or at least 31 genes, or at least 32 genes, or at least 33 genes, or at least 34 genes, or at least 35 genes, or at least 36 genes, or at least 37 genes, or at least 38 genes, or at least 39 genes, or at least 40 genes, or at least 41 genes, or at least 42 genes, or at least 43 genes, or at least 44 genes, or at least 45 genes, or at least 46 genes, or at least 47 genes, or at least 48 genes, or at least 49 genes, or at least 50 genes selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C.

In some aspects, the present disclosure provides a method of identifying a subject having acute myeloid leukemia (AML) that will be resistant to treatment with a combination of venetoclax and azacitidine, the method comprising: a) measuring the expression levels of at least 10 genes in a plurality of leukemia cells isolated from a biological sample from the subject, wherein the at least 10 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) identifying that the subject will be resistant to treatment with a combination of venetoclax and azacitidine when the percentage from step (c) is greater than the predetermined cutoff percentage.

In some aspects, the present disclosure provides a method of identifying a subject having acute myeloid leukemia (AML) that will be resistant to treatment with a combination of venetoclax and azacitidine, the method comprising: a) measuring the expression levels of at least 10 genes in a plurality of leukemia cells isolated from a biological sample from the subject, wherein the at least 10 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) identifying that the subject will be resistant to treatment with a combination of venetoclax and azacitidine when the percentage from step (c) is greater than the predetermined cutoff percentage.

In some aspects, the present disclosure provides a method of treating AML in a subject, the method comprising: a) measuring the expression levels of at least 10 genes in a plurality of leukemia cells isolated from a biological sample from the subject, wherein the at least 10 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) administering to the subject at least one therapeutically effective amount of at least one alternative therapy when the percentage from step (c) is greater than the predetermined cutoff percentage.

In some aspects, the present disclosure provides a method of treating AML in a subject, the method comprising: a) measuring the expression levels of at least 10 genes in a plurality of leukemia cells isolated from a biological sample from the subject, wherein the at least 10 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) administering to the subject at least one therapeutically effective amount of at least one therapy comprising a combination of venetoclax and azacitidine when the percentage from step (c) is less than the predetermined cutoff percentage.

In some aspects, the present disclosure provides a method of treating AML in a subject, the method comprising: a) measuring the expression levels of at least 10 genes in a plurality of leukemia cells isolated from a biological sample from the subject, wherein the at least 10 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) administering to the subject at least one therapeutically effective amount of at least one alternative therapy when the percentage from step (c) is greater than the predetermined cutoff percentage.

In some aspects, the present disclosure provides a method of treating AML in a subject, the method comprising: a) measuring the expression levels of at least 10 genes in a plurality of leukemia cells isolated from a biological sample from the subject, wherein the at least 10 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) administering to the subject at least one therapeutically effective amount of at least one therapy comprising a combination of venetoclax and azacitidine when the percentage from step (c) is less than the predetermined cutoff percentage.

In some aspects, the present disclosure provides a method of identifying a subject having acute myeloid leukemia (AML) that will be resistant to treatment with a combination of venetoclax and azacitidine, the method comprising: a) measuring the expression levels of at least 25 genes in a plurality of leukemia cells isolated from a biological sample from the subject, wherein the at least 25 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) identifying that the subject will be resistant to treatment with a combination of venetoclax and azacitidine when the percentage from step (c) is greater than the predetermined cutoff percentage.

In some aspects, the present disclosure provides a method of identifying a subject having acute myeloid leukemia (AML) that will be resistant to treatment with a combination of venetoclax and azacitidine, the method comprising: a) measuring the expression levels of at least 25 genes in a plurality of leukemia cells isolated from a biological sample from the subject, wherein the at least 25 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) identifying that the subject will be resistant to treatment with a combination of venetoclax and azacitidine when the percentage from step (c) is greater than the predetermined cutoff percentage.

In some aspects, the present disclosure provides a method of treating AML in a subject, the method comprising: a) measuring the expression levels of at least 25 genes in a plurality of leukemia cells isolated from a biological sample from the subject, wherein the at least 25 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) administering to the subject at least one therapeutically effective amount of at least one alternative therapy when the percentage from step (c) is greater than the predetermined cutoff percentage.

In some aspects, the present disclosure provides a method of treating AML in a subject, the method comprising: a) measuring the expression levels of at least 25 genes in a plurality of leukemia cells isolated from a biological sample from the subject, wherein the at least 25 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) administering to the subject at least one therapeutically effective amount of at least one alternative therapy when the percentage from step (c) is greater than the predetermined cutoff percentage.

In some aspects, the present disclosure provides a method of identifying a subject having acute myeloid leukemia (AML) that will be resistant to treatment with a combination of venetoclax and azacitidine, the method comprising: a) measuring the expression levels of at least 40 genes in a plurality of leukemia cells isolated from a biological sample from the subject, wherein the at least 40 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) identifying that the subject will be resistant to treatment with a combination of venetoclax and azacitidine when the percentage from step (c) is greater than the predetermined cutoff percentage.

In some aspects, the present disclosure provides a method of identifying a subject having acute myeloid leukemia (AML) that will be resistant to treatment with a combination of venetoclax and azacitidine, the method comprising: a) measuring the expression levels of at least 40 genes in a plurality of leukemia cells isolated from a biological sample from the subject, wherein the at least 40 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) identifying that the subject will be resistant to treatment with a combination of venetoclax and azacitidine when the percentage from step (c) is greater than the predetermined cutoff percentage.

In some aspects, the present disclosure provides a method of treating AML in a subject, the method comprising: a) measuring the expression levels of at least 40 genes in a plurality of leukemia cells isolated from a biological sample from the subject, wherein the at least 40 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) administering to the subject at least one therapeutically effective amount of at least one alternative therapy when the percentage from step (c) is greater than the predetermined cutoff percentage.

In some aspects, the present disclosure provides a method of treating AML in a subject, the method comprising: a) measuring the expression levels of at least 40 genes in a plurality of leukemia cells isolated from a biological sample from the subject, wherein the at least 40 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) administering to the subject at least one therapeutically effective amount of at least one alternative therapy when the percentage from step (c) is greater than the predetermined cutoff percentage.

In some aspects, the present disclosure provides a method of identifying a subject having acute myeloid leukemia (AML) that will be resistant to treatment with a combination of venetoclax and azacitidine, the method comprising: a) measuring the expression levels each of PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7 in a plurality of leukemia cells isolated from a biological sample from the subject; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) identifying that the subject will be resistant to treatment with a combination of venetoclax and azacitidine when the percentage from step (c) is greater than the predetermined cutoff percentage.

In some aspects, the present disclosure provides a method of identifying a subject having acute myeloid leukemia (AML) that will be resistant to treatment with a combination of venetoclax and azacitidine, the method comprising: a) measuring the expression levels each of AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C in a plurality of leukemia cells isolated from a biological sample from the subject; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) identifying that the subject will be resistant to treatment with a combination of venetoclax and azacitidine when the percentage from step (c) is greater than the predetermined cutoff percentage.

In some aspects, the present disclosure provides a method of treating AML in a subject, the method comprising: a) measuring the expression levels of each of PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7 in a plurality of leukemia cells isolated from a biological sample from the subject; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) administering to the subject at least one therapeutically effective amount of at least one alternative therapy when the percentage from step (c) is greater than the predetermined cutoff percentage.

In some aspects, the present disclosure provides a method of treating AML in a subject, the method comprising: a) measuring the expression levels of each of AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C in a plurality of leukemia cells isolated from a biological sample from the subject; b) classifying a measured cell as resistant to treatment with a combination of venetoclax and azacitidine or classifying a measured cell as responsive to treatment with a combination of venetoclax and azacitidine based on the expression levels measured in step (a); c) determining the percentage of leukemia cells in the plurality of leukemia cells that are classified as resistant to treatment with a combination of venetoclax and azacitidine; d) comparing the percentage from step (c) to a predetermined cutoff percentage; and e) administering to the subject at least one therapeutically effective amount of at least one alternative therapy when the percentage from step (c) is greater than the predetermined cutoff percentage.

In some aspects of the methods of the present disclosure, classifying a measured cell as resistant to treatment with a combination of venetoclax and azacitidine or classifying a measured cell as responsive to treatment with a combination of venetoclax and azacitidine based on the expression levels measured in step (a) comprises: i) determining a score based on the expression levels measured in step (a), wherein the score is determined using a machine learning classifier; and ii) classifying the cell as resistant to treatment with a combination of venetoclax and azacitidine based on the score.

In some aspects of the methods of the present disclosure, classifying a measured cell as resistant to treatment with a combination of venetoclax and azacitidine or classifying a measured cell as responsive to treatment with a combination of venetoclax and azacitidine based on the expression levels measured in step (a) comprises: i) determining a score based on the expression levels measured in step (a), wherein the score is determined using a machine learning classifier; ii) comparing the score determined in step (i) to a predetermined cutoff value; and iii) classifying the cell as resistant to treatment with a combination of venetoclax and azacitidine when the score is greater than or equal to the predetermined cutoff value or classifying the cell as responsive to treatment with a combination of venetoclax and azacitidine when the score is less than the predetermined cutoff value.

In some aspects of the methods of the present disclosure, classifying a measured cell as resistant to treatment with a combination of venetoclax and azacitidine or classifying a measured cell as responsive to treatment with a combination of venetoclax and azacitidine based on the expression levels measured in step (a) comprises: i) determining a score based on the expression level of the at least 10 genes, wherein the score is determined using a machine learning classifier; ii) comparing the score determined in step (i) to a predetermined cutoff value; and iii) classifying the cell as resistant to treatment with a combination of venetoclax and azacitidine when the score is less than or equal to the predetermined cutoff value or classifying the cell as responsive to treatment with a combination of venetoclax and azacitidine when the score is greater than the predetermined cutoff value.

In some aspects of the methods of the present disclosure, the machine learning classifier that is used to classify a measured cell as resistant to treatment with a combination of venetoclax and azacitidine or to classify a measured cell as responsive to treatment with a combination of venetoclax and azacitidine can be trained and validated using the expression levels of the genes measured in step (a) as measured in at least two training samples. In some aspects, at least one of the at least two training samples can comprise leukemia cells isolated from a subject that is responsive to treatment with venetoclax and azacitidine. In some aspects, at least one of the at least two training samples can comprise leukemia cells isolated from a subject that are resistant to treatment with venetoclax and azacitidine.

Thus, in a non-limiting example wherein at least 10 genes selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7 are measured in step (a) of the methods of the present disclosure, the machine learning classifier that is used to classify a measured cell as resistant to treatment with a combination of venetoclax and azacitidine or to classify a measured cell as responsive to treatment with a combination of venetoclax and azacitidine can be trained and validated using the expression levels of the at least 10 genes measured in at least two training samples, wherein at least one of the at least two training samples comprises leukemia cells isolated from a subject that is responsive to treatment with venetoclax and azacitidine, and wherein at least one of the at least two training samples comprises leukemia cells isolated from a subject that is resistant to treatment with venetoclax and azacitidine.

Thus, in another non-limiting example wherein at least 10 genes selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IF127, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C are measured in step (a) of the methods of the present disclosure, the machine learning classifier that is used to classify a measured cell as resistant to treatment with a combination of venetoclax and azacitidine or to classify a measured cell as responsive to treatment with a combination of venetoclax and azacitidine can be trained and validated using the expression levels of the at least 10 genes measured in at least two training samples, wherein at least one of the at least two training samples comprises leukemia cells isolated from a subject that is responsive to treatment with venetoclax and azacitidine, and wherein at least one of the at least two training samples comprises leukemia cells isolated from a subject that is resistant to treatment with venetoclax and azacitidine.

In some aspects, the machine learning classifier can be trained and validated using at least about 10 training samples, or a least about 20 training samples, or at least about 30 training samples, or at least about 40 training samples, or at least about 50 training samples, or at least about 60 training samples, or at least about 70 training samples, or at least about 80 training samples, or at least about 90 training samples, or at least about 90 training samples, or at least about 100 training samples, or at least about 250 training samples, or at least about 500 training samples or at least about 750 training samples, or at least about 1000 training samples, or at least about 10,000 training samples.

In some aspects of the methods of the present disclosure, a machine learning classifier can comprise a random forest model. In some aspects of the methods of the present disclosure, a machine learning classifier can comprise XGBoost (XGB), random forest (RF), Lasso and Elastic-Net Regularized Generalized Linear Models (glmnet), cforest, classification and regression tree (CART), treebag, k nearest-neighbor (knn), neural network (nnet), support vector machine radial (SVM-radial), support vector machine-linear (SVM-linear), naïve bayes (NB), multilayer perceptron (mlp) or any combination thereof.

In some aspects of the methods of the present disclosure, a plurality of leukemia cells can comprise at least about 10 leukemia cells, or at least about 50 leukemia cells, or at least about 100 leukemia cells, or at least about 150 leukemia cells, or at least about 200 leukemia cells, or at least about 250 leukemia cells, or at least about 300 leukemia cells, or at least about 350 leukemia cells, or at least about 400 leukemia cells, or at least about 450 leukemia cells, or at least about 500 leukemia cells, or at least about 750 leukemia cells, or at least about 1000 leukemia cells, or at least about 2500 leukemia cells, or at least about 5000 leukemia cells, or at least about 7500 leukemia cell, or at least about 10,000 leukemia cells.

In some aspects of the methods of the present disclosure, leukemia cells can comprise acute myeloid leukemia cells. In some aspects, leukemia cells can comprise acute myeloid leukemia blast cells. In some aspects, acute myeloid leukemia cells can comprise leukemia stem cells (LSCs). In some aspects, leukemia stem cells can comprise reactive oxygen species-low leukemia stem cells.

In some aspects of the preceding methods, the predetermined cutoff percentage can be at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%.

In some aspects of the preceding methods, the predetermined cutoff percentage can be at least about 25%.

In some aspects, an alternative therapy can comprise anti-cancer therapy, chemotherapy, targeted drug therapy, radiation therapy, immunotherapy, stem cell transplant or any combination thereof.

In some aspects of the methods of the present disclosure, an alternative therapy can comprise administering to the subject at least one therapeutically effective amount of at least one MCL-1 inhibitor. In some aspects of the methods of the present disclosure, an alternative therapy can comprise administering to the subject, at last one therapeutically effective amount of at least one MCL-1 inhibitor in combination with a therapeutically effective amount of venetoclax and a therapeutically effective amount of azacitidine. In some aspects of the present disclosure, an alternative therapy can comprise administering at least one therapeutically effective amount of at least one MCL-1 inhibitor in combination with a therapeutically effective amount of azacitidine.

In some aspects of the methods of the present disclosure, an alternative therapy can comprise administering to the subject, at last one therapeutically effective amount of at least one MCL-1 inhibitor in combination with a therapeutically effective amount of venetoclax and a therapeutically effective amount of decitabine. In some aspects of the present disclosure, an alternative therapy can comprise administering at least one therapeutically effective amount of at least one MCL-1 inhibitor in combination with a therapeutically effective amount of decitabine.

In some aspects of the methods of the present disclosure, an alternative therapy can comprise administering to the subject at least one therapeutically effective amount of an agent that targets CD70. In some aspects, an agent that targets CD70 can comprise an antibody, or an antigen-binding fragment thereof, that specifically binds to CD70. In some aspects, an agent that targets CD70 can comprise a CAR-T cell that specifically targets CD70. In some aspects, an alternative therapy can comprise administering to the subject at least one CD70-based immunotherapy.

In some aspects of the methods of the present disclosure, targeted drug therapy can comprise the administration of compounds that specifically target the cellular malfunctions that allow cancer cells to grow and proliferate. In some aspects of the methods of the present disclosure, targeted drug therapy can comprise administering to a subject a therapeutically effective amount of at least one agent that modulates a cellular pathway, wherein the cellular pathway is a pathway set forth in Table 1. In some aspects of the methods of the present disclosure, a targeted drug therapy can comprise administering to a subject a therapeutically effective amount of venetoclax in combination with a therapeutically effective amount of azacitidine.

In some aspects of the methods of the present disclosure, targeted drug therapy can comprise administering to a subject a therapeutically effective amount of an MCL-1 inhibitor. MCL-1 inhibitors can include, but are not limited to, YM155, VU103 or any combination thereof. Targeted drug therapy can comprising administering to a subject a therapeutically effective amount of an MCL-1 inhibitor in combination with a therapeutically effective amount of azacitidine. Targeted drug therapy can comprise administering to a subject a therapeutically effective amount of an MCL-1 inhibitor in combination with at least one hypomethylating agents. Hypomethylating agents can include, but are not limited to azacitidine, cytarabine, decitabine and any other hypomethylating agent known in the art. A metabolism modulating agent can be a BCL-2 inhibitor. BCL-2 inhibitors can include, but are not limited to, venetoclax, navitoclax, and any other BCL-2 inhibitor known in the art. In some aspects of any of the methods of the present disclosure, azacitidine can be substituted with at least one other hypomethylating agent, including, but not limited to azacitidine, cytarabine, decitabine and any other hypomethylating agent known in the art.

TABLE 1
Cellular Pathways

| Pathways | Pathways |
|---|---|
| Lysosome pathways | amino acid uptake pathways |
| nuclear import pathways | fatty acid oxidation pathways |
| PI3 Kinase and Akt signaling | myc signaling pathways |
| pathways | |
| NF-kB signaling pathways | Proteasome pathways |
| Autophagy pathways | nicotinamide metabolism |
| | (NAMPT enzyme, other |
| | regulators) pathways |
| Mitophagy pathways | amino acid catabolismo pathways |
| FIS1 signaling pathways | AMPK signaling pathways |
| CD38 pathways | protein synthesis pathways |
| nicotinamide metabolism pathways | Nampt pathways |
| Stress response pathways | Energy metabolism |
| DNA repair pathway | CD36 fatty acid oxidation |
| | pathway |
| DNA methylation | BCL-2 activity |

The present disclosure provides a method of providing an AML treatment recommendation for a subject, the method comprising: a) determining the expression level of at least one gene in a plurality of leukemia stem cells isolated from a biological sample from the subject, wherein the at least one gene is selected from NFκB, mTOR, RSK, ERK, MEK, stat3, src, mcl1; b) comparing the expression level of the at least one gene in the measured cells to a corresponding predetermined cutoff value; c) determining the percentage of leukemia cells in the plurality of leukemia cells that exhibit an expression level of the at least one gene that is greater than the corresponding predetermined cutoff value; d) comparing the percentage from step (c) to a predetermined cutoff percentage; and e) recommending a treatment comprising the administration of at least one therapeutically effective amount of at least one agent that targets the PI3K/AKT/mTOR pathway when the percentage from step (c) is greater than the predetermined cutoff percentage. In some aspects, the treatment recommendation can further comprise recommending the administration of at least one therapeutically effective amount of venetoclax, at least one therapeutically effective amount of azacitidine, at least one therapeutically effective amount of decitabine, at least one therapeutically effective amount of a combination of venetoclax and azacitidine, or at least one therapeutically effective amount of a combination of venetoclax and decitabine.

The present disclosure provides a method of treating AML in a subject, the method comprising: a) determining the expression level of at least one gene in a plurality of leukemia stem cells isolated from a biological sample from the subject, wherein the at least one gene is selected from NFκB, mTOR, RSK, ERK, MEK, stat3, src, mcl1; b) comparing the expression level of the at least one gene in the measured cells to a corresponding predetermined cutoff value; c) determining the percentage of leukemia cells in the plurality of leukemia cells that exhibit an expression level of the at least one gene that is greater than the corresponding predetermined cutoff value; d) comparing the percentage from step (c) to a predetermined cutoff percentage; and e) administering to the subject at least one therapeutically effective amount of at least one agent that targets the PI3K/AKT/mTOR pathway when the percentage from step (c) is greater than the predetermined cutoff percentage. In some aspects, the preceding method can further comprise administration of at least one therapeutically effective amount of venetoclax, at least one therapeutically effective amount of azacitidine, at least one therapeutically effective amount of decitabine, at least one therapeutically effective amount of a combination of venetoclax and azacitidine, or at least one therapeutically effective amount of a combination of venetoclax and decitabine.

The present disclosure provides a method of providing an AML treatment recommendation for a subject, the method comprising: a) determining the activation level of at least one gene in a plurality of leukemia stem cells isolated from a biological sample from the subject, wherein the at least one gene is selected from NFκB, mTOR, RSK, ERK, MEK, stat3, src, mcl1; b) comparing the activation level of the at least one gene in the measured cells to a corresponding predetermined cutoff value; c) determining the percentage of leukemia cells in the plurality of leukemia cells that exhibit an activation level of the at least one gene that is greater than the corresponding predetermined cutoff value; d) comparing the percentage from step (c) to a predetermined cutoff percentage; and e) recommending a treatment comprising the administration of at least one therapeutically effective amount of at least one agent that targets the PI3K/AKT/mTOR pathway when the percentage from step (c) is greater than the predetermined cutoff percentage. In some aspects, the treatment recommendation can further comprise recommending the administration of at least one therapeutically effective amount of venetoclax, at least one therapeutically effective amount of azacitidine, at least one therapeutically effective amount of decitabine, at least one therapeutically effective amount of a combination of venetoclax and azacitidine, or at least one therapeutically effective amount of a combination of venetoclax and decitabine.

The present disclosure provides a method of treating AML in a subject, the method comprising: a) determining the activation level of at least one gene in a plurality of leukemia stem cells isolated from a biological sample from the subject, wherein the at least one gene is selected from NFκB, mTOR, RSK, ERK, MEK, stat3, src, mcl1; b) comparing the activation level of the at least one gene in the measured cells to a corresponding predetermined cutoff value; c) determining the percentage of leukemia cells in the plurality of leukemia cells that exhibit an activation level of the at least one gene that is greater than the corresponding predetermined cutoff value; d) comparing the percentage from step (c) to a predetermined cutoff percentage; and e) administering to the subject at least one therapeutically effective amount of at least one agent that targets the PI3K/AKT/mTOR pathway when the percentage from step (c) is greater than the predetermined cutoff percentage. In some aspects, the preceding method can further comprise administration of at least one therapeutically effective amount of venetoclax, at least one therapeutically effective amount of azacitidine, at least one therapeutically effective amount of decitabine, at least one therapeutically effective amount of a combination of venetoclax and azacitidine, or at least one therapeutically effective amount of a combination of venetoclax and decitabine.

The present disclosure provides a method of providing an AML treatment recommendation for a subject, the method comprising: a) determining the phosphorylation level of at least one gene in a plurality of leukemia stem cells isolated from a biological sample from the subject, wherein the at least one gene is selected from NFκB, mTOR, RSK, ERK, MEK, stat3, src, mcl1; b) comparing the phosphorylation level of the at least one gene in the measured cells to a corresponding predetermined cutoff value; c) determining the percentage of leukemia cells in the plurality of leukemia cells that exhibit an phosphorylation level of the at least one gene that is greater than the corresponding predetermined cutoff value; d) comparing the percentage from step (c) to a predetermined cutoff percentage; and e) recommending a treatment comprising the administration of at least one therapeutically effective amount of at least one agent that targets the PI3K/AKT/mTOR pathway when the percentage from step (c) is greater than the predetermined cutoff percentage. In some aspects, the treatment recommendation can further comprise recommending the administration of at least one therapeutically effective amount of venetoclax, at least one therapeutically effective amount of azacitidine, at least one therapeutically effective amount of decitabine, at least one therapeutically effective amount of a combination of venetoclax and azacitidine, or at least one therapeutically effective amount of a combination of venetoclax and decitabine.

The present disclosure provides a method of treating AML in a subject, the method comprising: a) determining the phosphorylation level of at least one gene in a plurality of leukemia stem cells isolated from a biological sample from the subject, wherein the at least one gene is selected from NFκB, mTOR, RSK, ERK, MEK, stat3, src, mcl1; b) comparing the phosphorylation level of the at least one gene in the measured cells to a corresponding predetermined cutoff value; c) determining the percentage of leukemia cells in the plurality of leukemia cells that exhibit an phosphorylation level of the at least one gene that is greater than the corresponding predetermined cutoff value; d) comparing the percentage from step (c) to a predetermined cutoff percentage; and e) administering to the subject at least one therapeutically effective amount of at least one agent that targets the PI3K/AKT/mTOR pathway when the percentage from step (c) is greater than the predetermined cutoff percentage. In some aspects, the preceding method can further comprise administration of at least one therapeutically effective amount of venetoclax, at least one therapeutically effective amount of azacitidine, at least one therapeutically effective amount of decitabine, at least one therapeutically effective amount of a combination of venetoclax and azacitidine, or at least one therapeutically effective amount of a combination of venetoclax and decitabine.

In some aspects of the preceding methods, a predetermined cutoff percentage can be at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%. In some aspects, a predetermined cutoff percentage can be at least about 75%.

In some aspects of the methods of the present disclosure, an agent that targets the PI3K/AKT/mTor pathway can be an agent that inhibits at least one of PI3K, AKT and mTOR.

In some aspects of the methods of the present disclosure, an agent that targets the PI3K/AKT/mTOR pathway can comprise, but is not limited to, everolimus, temsirolimus, sirolimus, CC-223, vistusertib, nab-rapamycin, CC-115, sapanisertib, copanlisib, duvelisib, alpelisib, idelalisib, puquitinib, leniolisib, buparlisib, RTB101, umbralisib, TG-100-115, nemiralisib, GSK2636771, fimepinostat, tenalisib, serabelisib, INCB50465, SF1126, GDC-0077, AZD8186, ME401, IPI-549, MEN 1611, ASN003, bimiralisib, GDC0084, voxtalisib, LY3023414, gedatolisib, ARG-092, MK-2206, iapatasertib, uprosertib, capivasertib, triciribine, ARQ-751, PF-04979064 and PF-04691502.

The present disclosure provides a method of providing an AML treatment recommendation for a subject, the method comprising: a) determining the expression level of at least one gene in a plurality of leukemia stem cells isolated from a biological sample from the subject, wherein the at least one gene is selected from CD38, LAMPS, SLC44A1 (CD92), PLAC8, NCAM1 (CD56) and CD70; b) comparing the expression level of the at least one gene in the measured cells to a corresponding predetermined cutoff value; c) determining the percentage of leukemia cells in the plurality of leukemia cells that exhibit an expression level of the at least one gene that is greater than the corresponding predetermined cutoff value; d) comparing the percentage from step (c) to a predetermined cutoff percentage; e) recommending a treatment comprising the administration of at least one therapeutically effective amount of at least one agent that targets the at least one gene when the percentage from step (c) is greater than the predetermined cutoff percentage. In some aspects, the treatment recommendation can further comprise recommending the administration of at least one therapeutically effective amount of venetoclax, at least one therapeutically effective amount of azacitidine, at least one therapeutically effective amount of decitabine, at least one therapeutically effective amount of a combination of venetoclax and azacitidine, or at least one therapeutically effective amount of a combination of venetoclax and decitabine.

The present disclosure provides a method of treating AML in a subject, the method comprising: a) determining the expression level of at least one gene in a plurality of leukemia stem cells isolated from a biological sample from the subject, wherein the at least one gene is selected from CD38, LAMPS, SLC44A1 (CD92), PLAC8, NCAM1 (CD56) and CD70; b) comparing the expression level of the at least one gene in the measured cells to a corresponding predetermined cutoff value; c) determining the percentage of leukemia cells in the plurality of leukemia cells that exhibit an expression level of the at least one gene that is greater than the corresponding predetermined cutoff value; d) comparing the percentage from step (c) to a predetermined cutoff percentage; e) administering to the subject at least one therapeutically effective amount of at least one agent that targets the at least one gene when the percentage from step (c) is greater than the predetermined cutoff percentage. In some aspects, the preceding method can further comprise administration of at least one therapeutically effective amount of venetoclax, at least one therapeutically effective amount of azacitidine, at least one therapeutically effective amount of decitabine, at least one therapeutically effective amount of a combination of venetoclax and azacitidine, or at least one therapeutically effective amount of a combination of venetoclax and decitabine.

In some aspects of the preceding methods, a predetermined cutoff percentage can be at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%. In some aspects, a predetermined cutoff percentage can be at least about 75%.

In some aspects of the preceding methods, an at least one agent that targets the at least one gene can directly target the at least one gene, including, but not limited to, inhibiting the at least one gene, decreasing the expression of the at least one gene, etc. In some aspects of the preceding methods, an at least one agent that targets the at least one gene can indirectly target the at least one gene. Indirectly targeting the at least one gene can include, but is not limited to, targeting upstream regulators and/or downstream effectors of the at least one gene.

In some aspects of the preceding methods, the at least one gene can be CD38. In aspects wherein the at least one gene is CD38, the at least one agent that targets the at least one gene (CD38) can comprise, but is not limited to, daratumumab.

In some aspects of the preceding methods, the at least one gene can be LAMPS. In aspects wherein the at least one gene is LAMPS, the at least one agent that targets the at least one gene (LAMPS) can comprise, but is not limited to, pinometostat.

In some aspects of the preceding methods, the at least one gene can be PLAC8. In aspects wherein the at least one gene is PLAC8, the at least one agent that targets the at least one gene (PLAC8) can comprise, but is not limited to, any PI3K inhibitor known in the art. Non-limiting examples of PI3K inhibitors include, but are not limited to, copanlisib, duvelisib, alpelisib, idelalisib, puquitinib, leniolisib, buparlisib, RTB101, umbralisib, TG-100-115, nemiralisib, GSK2636771, fimepinostat, tenalisib, serabelisib, INCB50465, SF1126, GDC-0077, AZD8186, ME401, IPI-549, MEN 1611 and ASN003.

In some aspects of the preceding methods, the at least one gene can be NCAM1 (CD56). In aspects wherein the at least one gene is NCAM1, the at least one agent that targets the at least one gene (NCAM1) can comprise, but is not limited to, lorvotuzumab or mertansine.

In some aspects of the preceding methods, the at least one gene can be SLC44A1 (CD92). In aspects wherein the at least one gene is SLC44A1, the at least one agent that targets the at least one gene (SLC44A1) can comprise, but is not limited to, an antibody or CAT-T cell that specifically binds to SLC44A1.

In some aspects of the preceding methods, the at least one gene can be SLC44A1 (CD92). In aspects wherein the at least one gene is SLC44A1, the at least one agent that targets the at least one gene (SLC44A1) can comprise, but is not limited to, an antibody (or antigen-binding fragment thereof) or CAR-T cell that specifically binds to SLC44A1.

In some aspects of the preceding methods, the at least one gene can be CD70. In aspects wherein the at least one gene is CD70, the at least one agent that targets the at least one gene (CD70) can comprise, but is not limited to, an antibody (or antigen-binding fragment thereof) or CAR-T cell that specifically binds to CD70.

The present disclosure provides a method of treating AML in a subject comprising administering to the subject a combination of at least one therapeutically effective amount of at least one BCL-2 inhibitor and at least one therapeutically effective amount of at least one agent that targets the PI3K/AKT/mTOR pathway. The present disclosure provides a method of treating AML in a subject comprising administering to the subject a combination of at least one therapeutically effective amount of venetoclax and at least one therapeutically effective amount of at least one agent that targets the PI3K/AKT/mTOR pathway.

The present disclosure provides a combination of at least one BCL-2 inhibitor and at least one agent that targets the PI3K/AKT/mTOR pathway for use in a method for treating AML. The present disclosure provides a combination of venetoclax and at least one agent that targets the PI3K/AKT/mTOR pathway for use in a method for treating AML.

The present disclosure provides at least one BCL-2 inhibitor for use in a method for treating AML, wherein the method further comprises the administration of at least one agent that targets the PI3K/AKT/mTOR pathway. The present disclosure provides venetoclax for use in a method for treating AML, wherein the method further comprises the administration of at least one agent that targets the PI3K/AKT/mTOR pathway.

The present disclosure provides at least one agent that targets the PI3K/AKT/mTOR pathway for use in a method for treating AML, wherein the method further comprises the administration of at least one BCL-2 inhibitor. The present disclosure provides at least one agent that targets the PI3K/AKT/mTOR pathway for use in a method for treating AML, wherein the method further comprises the administration of venetoclax.

The present disclosure provides the use of a combination of at least one BCL-2 inhibitor and at least one agent that targets the PI3K/AKT/mTOR pathway in the manufacture of a medicament for the treatment of AML. The present disclosure provides the use of a combination of venetoclax and at least one agent that targets the PI3K/AKT/mTOR pathway in the manufacture of a medicament for the treatment of AML.

The present disclosure provides a method of treating AML in a subject comprising administering to the subject a combination of at least one therapeutically effective amount of at least one BCL-2 inhibitor, at least one therapeutically effective amount of at least one agent that targets the PI3K/AKT/mTOR pathway and at least one therapeutically effective amount of at least one hypomethylating agent. The present disclosure provides a method of treating AML in a subject comprising administering to the subject a combination of at least one therapeutically effective amount of venetoclax, at least one therapeutically effective amount of at least one agent that targets the PI3K/AKT/mTOR pathway and at least one therapeutically effective amount of azacitidine.

The present disclosure provides the use of a combination of at least one BCL-2 inhibitor, at least one agent that targets the PI3K/AKT/mTOR pathway and at least one hypomethylating agent for use in a method for treating AML. The present disclosure provides the use of a combination of venetoclax, at least one agent that targets the PI3K/AKT/mTOR pathway, and azacitidine for use in a method for treating AML.

The present disclosure provides at least one BCL-2 inhibitor for use in a method for treating AML, wherein the method further comprises the administration of at least one agent that targets the PI3K/AKT/mTOR pathway and the administration of at least one hypomethylating agent. The present disclosure provides venetoclax for use in a method for treating AML, wherein the method further comprises the administration of at least one agent that targets the PI3K/AKT/mTOR pathway and the administration of azacitidine.

The present disclosure provides at least one agent that targets the PI3K/AKT/mTOR pathway for use in a method for treating AML, wherein the method further comprises the administration of at least one BCL-2 inhibitor and the administration of at least one hypomethylating agent. The present disclosure provides at least one agent that targets the PI3K/AKT/mTOR pathway for use in a method for treating AML, wherein the method further comprises the administration of venetoclax and the administration of azacitidine.

The present disclosure provides at least one hypomethylating agent for use in a method for treating AML, wherein the method further comprises the administration of at least one agent that targets the PI3K/AKT/mTOR pathway and the administration of at least one BCL-2 inhibitor. The present disclosure provides azacitidine for use in a method for treating AML, wherein the method further comprises the administration of at least one agent that targets the PI3K/AKT/mTOR pathway and the administration of venetoclax.

The present disclosure provides the use of a combination of at least one BCL-2 inhibitor, at least one agent that targets the PI3K/AKT/mTOR pathway and at least one hypomethylating agent in the manufacture of a medicament for the treatment of AML. The present disclosure provides the use of a combination of venetoclax, at least one agent that targets the PI3K/AKT/mTOR pathway, and azacitidine in the manufacture of a medicament for the treatment of AML.

In some aspects of the methods of the present disclosure, determining the expression level of a gene, or of a plurality of genes, can comprise PCR, high-throughput sequencing, next generation sequencing, RNA-sequencing, Northern Blot, reverse transcription PCR (RT-PCR), real-time PCR (qPCR), quantitative PCR, qRT-PCR, flow cytometry, mass spectrometry, microarray analysis, digital droplet PCR, Western Blot or any combination thereof. In some aspects, RNA-sequence can comprise Cellular Indexing of Transcriptomes and Epitopes by Sequencing (CITE-Seq).

In some aspects of the methods of the present disclosure, determining the activation level and/or the phosphorylation level can comprise Western Blot, flow cytometry, mass spectrometry, phosphoflow analysis, as would be appreciated by the skilled artisan.

In some aspects of the methods of the present disclosure, a biological sample can comprise blood, a bone marrow biopsy, a bone marrow aspirate, a biopsy of a chloroma, a tissue biopsy, cerebrospinal fluid or any combination thereof.

Samples can be isolated from a subject using methods known in the art. In a non-limiting example, a cerebrospinal fluid sample can be isolated from a subject by performing a lumbar puncture (spinal tap). In another non-limiting example, a bone marrow biopsy or a bone marrow aspirate can be isolated by using a needle to pierce a bone, such as a hip bone, to obtain bone marrow.

In some aspects of the methods of the present disclosure, a subject can have been previously diagnosed with acute myeloid leukemia. In some aspects, a subject can have been previously administered an initial therapy. The subject may have not responded to the initial therapy or may have only partially responded to the initial therapy. In some aspects, a subject can have relapsed acute myeloid leukemia.

In some aspects, a response to a therapy in a subject can be evaluated using methods known in the art. In a non-limiting example, a response to a therapy can be evaluated by isolating a sample from the subject (be plasma, serum, blood, bone marrow biopsy, a bone marrow aspirate, a biopsy of a chloroma, a tissue biopsy, cerebrospinal fluid or any combination thereof) and analyzing the sample to determine the concentration of leukemia cells, markers or combination thereof.

In some aspects of the methods of the present disclosure, an initial therapy can comprise administering to the subject a therapeutically effective amount of venetoclax in combination with a therapeutically effective amount of azacitidine. In some aspects of the methods of the present disclosure, an initial therapy can comprise administering to a subject a therapeutically effective amount of an anti-cancer therapy, chemotherapy, targeted drug therapy, radiation therapy, immunotherapy, stem cell transplant or any combination thereof.

In some aspects of the methods of the present disclosure, a subject can be at least about 5 years of age, or at least about 10 years of age, or at least about 15 years of age, or at least about 18 years of age, or at least about 20 years of age, or at least about 25 years of age, or at least about 30 years of age, or at least about 35 years of age, or at least about 40 years of age, or at least about 45 years of age, or at least about 50 years of age, or at least about 55 years of age, or at least about 60 years of age, or at least about 65 years of age, or at least about 70 years of age, or at least about 75 years of age, or at least about 80 years of age, or at least about 85 years of age, or at least about 90 years of age, or at least about 95 years of age, or at least about 100 years of age.

In some aspects, a predetermined cutoff value or a predetermined cutoff percentage can have a negative predictive value of at least about 80%, or at least about 85%, or at least about 90%, or at least about 95%, or at least about 99%, or at least about 99.9%.

In some aspects, a predetermined cutoff value or a predetermined cutoff percentage can have a positive predictive value of at least about 80%, or at least about 85%, or at least about 90%, or at least about 95%, or at least about 99%, or at least about 99.9%.

In some aspects, a predetermined cutoff value or a predetermined cutoff percentage can have a sensitivity of at least about 80%, or at least about 85%, or at least about 90%, or at least about 95%, or at least about 99%, or at least about 99.9%.

In some aspects, a predetermined cutoff value or a predetermined cutoff percentage can have a specificity of at least about 80%, or at least about 85%, or at least about 90%, or at least about 95%, or at least about 99%, or at least about 99.9%.

In some aspects of the methods of the present disclosure, immunotherapy can comprise administering a therapeutically effective amount of at least one antibody, at least one checkpoint inhibitor, at least one chimeric antigen receptor-modified T-Cell (CAR-T cell, or any combination thereof. Immunotherapy can comprise adoptive cell transfer therapy. In some aspects of the methods of the present disclosure, immunotherapy can comprise administering a therapeutically effective amount of at least one antibody, wherein the at least one antibody binds to at least one AML, cell surface protein. In some aspects of the methods of the present disclosure, immunotherapy can comprise administering a therapeutically effective amount of at least one antibody, wherein the at least one antibody binds specifically to at least one AML cell surface protein.

In some aspects of the methods of the present disclosure, immunotherapy can comprise administering checkpoint inhibitors. Checkpoint inhibitors can comprise antibodies. Checkpoint inhibitors include, but are not limited to, anti-CTLA4 antibodies, anti-PD-1 antibodies, anti-PD-L1 antibodies, anti-A2AR antibodies, anti-B7-H3 antibodies, anti-B7-H4 antibodies, anti-BTLA antibodies, anti-IDO antibodies, anti-KIR antibodies, anti-LAG3 antibodies, anti-TIM3 antibodies and anti-VISTA (V-domain Ig suppressor of T cell activation) antibodies.

Anti-CTLA4 antibodies can include, but are not limited to, ipilimumab, tremelimumab and AGEN-1884. Anti-PD-1 antibodies include, but are not limited to, pembrolizumab, nivolumab pidilizumab, cemiplimab, REGN2810, AMP-224, MEDI0680, PDR001 and CT-001. Anti-PD-L1 antibodies include, but are not limited to atezolizumab, avelumab and durvalumab. Anti-CD137 antibodies include, but are not limited to, urelumab. Anti-B7-H3 antibodies include, but are not limited to, MGA271. Anti-KIR antibodies include, but are not limited to, Lirilumab. Anti-LAG3 antibodies include, but are not limited to, BMS-986016.

The term “immunotherapy” can refer to activating immunotherapy or suppressing immunotherapy. As will be appreciated by those in the art, activating immunotherapy refers to the use of a therapeutic agent that induces, enhances, or promotes an immune response, including, e.g., a T cell response while suppressing immunotherapy refers to the use of a therapeutic agent that interferes with, suppresses, or inhibits an immune response, including, e.g., a T cell response. Activating immunotherapy may comprise the use of checkpoint inhibitors. Activating immunotherapy may comprise administering to a subject a therapeutic agent that activates a stimulatory checkpoint molecule. Stimulatory checkpoint molecules include, but are not limited to, CD27, CD28, CD40, CD122, CD137, OX40, GITR and ICOS. Therapeutic agents that activate a stimulatory checkpoint molecule include, but are not limited to, MEDI0562, TGN1412, CDX-1127, lipocalin.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody that binds to a target refers to an antibody that is capable of binding the target with sufficient affinity such that the antibody is useful as a diagnostic and/or therapeutic agent in targeting the target. In one embodiment, the extent of binding of an anti-target antibody to an unrelated, non-target protein is less than about 10% of the binding of the antibody to target as measured, e.g., by a radioimmunoassay (RIA) or biacore assay. In certain embodiments, an antibody that binds to a target has a dissociation constant (Kd) of <1 μM, <100 nM, <10 nM, <1 nM, <0.1 nM, <0.01 nM, or <0.001 nM (e.g. 10⁸ M or less, e.g. from 10⁸ M to 10¹³ M, e.g., from 10⁹ M to 10¹³ M). In certain embodiments, an anti-target antibody binds to an epitope of a target that is conserved among different species.

A “blocking antibody” or an “antagonist antibody” is one that partially or fully blocks, inhibits, interferes, or neutralizes a normal biological activity of the antigen it binds. For example, an antagonist antibody may block signaling through an immune cell receptor (e.g., a T cell receptor) so as to restore a functional response by T cells (e.g., proliferation, cytokine production, target cell killing) from a dysfunctional state to antigen stimulation.

An “agonist antibody” or “activating antibody” is one that mimics, promotes, stimulates, or enhances a normal biological activity of the antigen it binds. Agonist antibodies can also enhance or initiate signaling by the antigen to which it binds. In some embodiments, agonist antibodies cause or activate signaling without the presence of the natural ligand. For example, an agonist antibody may increase memory T cell proliferation, increase cytokine production by memory T cells, inhibit regulatory T cell function, and/or inhibit regulatory T cell suppression of effector T cell function, such as effector T cell proliferation and/or cytokine production.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

CAR-T cells are T cells that are genetically modified to stably express at least one chimeric antigen receptor (CAR). A CAR can comprise an extracellular domain, transmembrane domain and a cytoplasmic domain. A CAR can comprise an antigen binding domain. An antigen binding domain can be located in an extracellular domain. In some aspects of the methods of the present disclosure, the antigen binding domain binds to at least one AML cell surface protein. In some aspects of the methods of the present disclosure, the antigen binding domain binds to CD7. A CAR can also comprise an extracellular spacer (hinge) domain. An extracellular spacer can be located in an extracellular domain. A CAR can comprise a signaling domain. A signaling domain can be a T-cell activation domain. A signaling domain can be located in a cytoplasmic domain. A CAR can comprise at least one costimulatory domain. A CAR can comprise at least two costimulatory domains. A CAR can comprise at least three costimulatory domains. A costimulatory domain can be located in a cytoplasmic domain.

In some aspects of the methods of the present disclosure CAR-T cells can be autologous with respect to a subject. In some aspects, CAR-T cells can be allogeneic with respect to a subject.

In some aspects of the methods of the present disclosure, CAR-T cells may be administered either alone, or as a pharmaceutical composition in combination with diluents and/or with other components such as IL-2 or other cytokines or cell populations. Briefly, pharmaceutical compositions can comprise a plurality of CAR-T cells in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. CAR-T cells and related compositions can be administered to a subject intravenously.

A CAR-T cell can comprise a chimeric antigen receptor. A chimeric antigen receptor can comprise an antigen binding domain. An antigen binding domain can bind to CD7.

A first therapy can comprise administering to the subject a therapeutically effective amount of an immunotherapy, a stem cell transplant, anti-cancer therapy, chemotherapy, targeted drug therapy, radiation therapy, or any combination thereof.

In methods of the present disclosure, venetoclax may be administered orally. Venetoclax may be administered in a ramp-up schedule fashion over the course of 5 weeks, wherein during the first week 20 mg of venetoclax is administered daily, during the second week 50 mg of venetoclax is administered daily, during the third week 100 mg of venetoclax is administered daily, during the fourth week 200 mg of venetoclax is administered daily and during the fifth week and onwards until the end of treatment 400 mg of venetoclax is administered daily (final dose amount). Alternatively, the final dose of venetoclax can be about 300 to about 1400 mg daily. The final dose amount of venetoclax can be 400 mg daily. Alternatively, the final dose amount of venetoclax can be 800 mg daily. Alternatively still, the final dose amount of venetoclax can be 1200 mg daily. During the ramp-up schedule, the dose of venetoclax administered during any of the first, second, third or fourth weeks can be adjusted to be about 20 mg, about 50 mg, about 100 mg and about 200 mg respectively.

Azacitidine can be administered intravenously or subcutaneously. Azacitidine can be administered at a concentration of about 75 mg/m² daily for about 7 days about every 4 weeks. Alternatively, Azacitidine can be administered at a concentration of about 100 mg/m² daily for about 7 days about every 4 weeks.

In alternative aspects, Azacitidine can be administered orally. Azacitidine can be administered orally at a concentration of about 10 mg, or about 25 mg, or about 50 mg, or about 75 mg, or about 100 mg, or about 120 mg, or about 150 mg, or about 200 mg, or about 250 mg, or about 300 mg, or about 350 mg, or about 400 mg, or about 450 mg, or about 480 mg, or about 500 mg, or about 550 mg, or about 600 mg daily for about 7 days about every 4 weeks, or about 14 days about every 4 weeks, or about 21 days about every 4 weeks.

The terms “effective amount” and “therapeutically effective amount” of an agent or compound are used in the broadest sense to refer to a nontoxic but sufficient amount of an active agent or compound to provide the desired effect or benefit.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, and leukemia. More particular examples of such cancers include adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer or cervical cancer.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The term “refractory” as used herein, is used in its broadest sense to refer to instances in which the disease present in a subject does not respond to a particular therapy, i.e. the therapy provides no or decreased clinical benefit to that particular subject.

### EXEMPLARY EMBODIMENTS

1. A method of identifying a subject having acute myeloid leukemia (AML) that will be resistant to treatment with a combination of venetoclax and azacitidine, the method comprising:
a) measuring the expression levels of at least 10 genes in a plurality of leukemia cells isolated from a biological sample from the subject, wherein the at least 10 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7; or AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRS S2, PRTN3, RNASE1, S100A8, S100A9, S100A12, SERPINB2, TCN1, THBS1, TOP2A, TPSAB1, TPSB2, and UBE2C;
b) classifying a measured cell as resistant to treatment with a combination of venetoclax and azacitidine or classifying a measured cell as responsive to treatment with a combination of venetoclax and azacitidine based on the expression levels measured in step (a);
c) determining the percentage of leukemia cells in the plurality of leukemia cells that are classified as resistant to treatment with a combination of venetoclax and azacitidine;
d) comparing the percentage from step (c) to a predetermined cutoff percentage; and
e) identifying that the subject will be resistant to treatment with a combination of venetoclax and azacitidine when the percentage from step (c) is greater than the predetermined cutoff percentage.

2. The method of exemplary embodiment 1, wherein classifying a measured cell as resistant to treatment with a combination of venetoclax and azacitidine or classifying a measured cell as responsive to treatment with a combination of venetoclax and azacitidine based on the expression levels measured in step (a) comprises:
i) determining a score based on the expression level of the at least 10 genes, wherein the score is determined using a machine learning classifier;
ii) comparing the score determined in step (i) to a predetermined cutoff value; and
iii) classifying the cell as resistant to treatment with a combination of venetoclax and azacitidine when the score is greater than or equal to the predetermined cutoff value or classifying the cell as responsive to treatment with a combination of venetoclax and azacitidine when the score is less than the predetermined cutoff value.

3. The method of exemplary embodiment 1, wherein classifying a measured cell as resistant to treatment with a combination of venetoclax and azacitidine or classifying a measured cell as responsive to treatment with a combination of venetoclax and azacitidine based on the expression levels measured in step (a) comprises:
i) determining a score based on the expression level of the at least 10 genes, wherein the score is determined using a machine learning classifier;
ii) comparing the score determined in step (i) to a predetermined cutoff value; and
iii) classifying the cell as resistant to treatment with a combination of venetoclax and azacitidine when the score is less than or equal to the predetermined cutoff value or classifying the cell as responsive to treatment with a combination of venetoclax and azacitidine when the score is greater than the predetermined cutoff value.

4. The method of exemplary embodiment 2 or exemplary embodiment 3, wherein the machine learning classifier is trained and validated using the expression levels of the at least 10 genes measured in at least two training samples, wherein at least one of the at least two training samples comprises leukemia cells isolated from a subject that is responsive to treatment with venetoclax and azacitidine, and wherein at least one of the at least two training samples comprises leukemia cells isolated from a subject that is resistant to treatment with venetoclax and azacitidine.

5. The method of any one of the preceding exemplary embodiments, wherein step (a) comprises measuring the expression levels of at least 25 genes in the plurality of leukemia cells, wherein the at least 25 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7; or AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C.

6. The method of any one of the preceding exemplary embodiments, wherein step (a) comprises measuring the expression levels of at least 40 genes in the plurality of leukemia cells, wherein the at least 40 genes are selected from PCDH9, LAMPS, PPP1R27, MPO, CTSG, RNASE1, AREG, VCAN, S100A9, S100A8, MT2A, ELANE, RNASE3, RETN, RND3, FCER1A, AGR2, FN1, MKI67, TPSB2, U2AF1, FAM83A, IFIT2, PLBD1, S100A12, PRTN3, DLK1, MT1G, THBS1, GOS2, TPSAB1, LINC00861, HPGD, C1QA, HMOX1, CCL4, SERPINB2, CCL4L2, MS4A2, DDIT4L, MT1H, FCGR3A, C1QB, CLC, MMP9, PRG2, HDC, C1QC, CCL2 and CCL7; or AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C.

7. The method of any of the preceding exemplary embodiments, wherein the plurality of leukemia cells comprises at least about 300 leukemia cells.

8. The method of any one of the preceding exemplary embodiments, wherein the leukemia cells comprise acute myeloid leukemia blast cells.

9. The method of any one of the preceding exemplary embodiments, wherein the leukemia cells comprise leukemia stem cells.

10. The method of exemplary embodiment 9, wherein the leukemia stem cells comprise reactive oxygen species-low leukemia stem cells.

11. The method of any one of the preceding exemplary embodiments, wherein the predetermined cutoff percentage is at least about 25%.

12. The method of any of the preceding exemplary embodiments, further comprising providing a treatment recommendation to the subject that is identified as a subject that is resistant to treatment with a combination of venetoclax and azacitidine, wherein the treatment recommendation comprises recommending the administration of at least one therapeutically effective amount of at least one alternative therapy.

13. The method of any of the preceding exemplary embodiments, further comprising administering to the subject identified as resistant to treatment with a combination of venetoclax and azacitidine at least one therapeutically effective amount of at least one alternative therapy.

14. The method of exemplary embodiment 12 or exemplary embodiment 13, wherein the at least one alternative therapy comprises anti-cancer therapy, chemotherapy, targeted drug therapy, radiation therapy, immunotherapy, stem cell transplant or any combination thereof.

15. A method of providing an AML treatment recommendation for a subject, the method comprising:
a) determining the expression level of at least one gene in a plurality of leukemia stem cells isolated from a biological sample from the subject, wherein the at least one gene is selected from NFκB, mTOR, RSK, ERK, MEK, stat3, src, mcl1;
b) comparing the expression level of the at least one gene in the measured cells to a corresponding predetermined cutoff value;
c) determining the percentage of leukemia cells in the plurality of leukemia cells that exhibit an expression level of the at least one gene that is greater than the corresponding predetermined cutoff value;
d) comparing the percentage from step (c) to a predetermined cutoff percentage; and
e) recommending a treatment comprising the administration of at least one therapeutically effective amount of at least one agent that targets the PI3K/AKT/mTOR pathway when the percentage from step (c) is greater than the predetermined cutoff percentage.

16. The method of exemplary embodiment 15, wherein the at least one agent that targets the PI3K/AKT/mTOR is an agent that inhibits at least one of PI3K, AKT and mTOR.

17. The method of exemplary embodiment 15, wherein the at least one agent that targets the PI3K/AKT/mTOR pathway is selected from everolimus, temsirolimus, sirolimus, CC-223, vistusertib, nab-rapamycin, CC-115, sapanisertib, copanlisib, duvelisib, alpelisib, idelalisib, puquitinib, leniolisib, buparlisib, RTB 101, umbralisib, TG-100-115, nemiralisib, GSK2636771, fimepinostat, tenalisib, serabelisib, INCB50465, SF1126, GDC-0077, AZD8186, ME401, IPI-549, MEN 1611, ASN003, bimiralisib, GDC0084, voxtalisib, LY3023414, gedatolisib, ARG-092, MK-2206, iapatasertib, uprosertib, capivasertib, triciribine, ARQ-751, PF-04979064 and PF-04691502.

18. A method of providing an AML treatment recommendation for a subject, the method comprising:
a) determining the expression level of at least one gene in a plurality of leukemia stem cells isolated from a biological sample from the subject, wherein the at least one gene is selected from CD38, LAMPS, SLC44A1 (CD92), PLAC8, NCAM1 (CD56) and CD70;
b) comparing the expression level of the at least one gene in the measured cells to a corresponding predetermined cutoff value;
c) determining the percentage of leukemia cells in the plurality of leukemia cells that exhibit an expression level of the at least one gene that is greater than the corresponding predetermined cutoff value;
d) comparing the percentage from step (c) to a predetermined cutoff percentage;
e) recommending a treatment comprising the administration of at least one therapeutically effective amount of at least one agent that targets the at least one gene when the percentage from step (c) is greater than the predetermined cutoff percentage.

19. The method of exemplary embodiment 18, wherein the at least one gene is CD38.

20. The method of exemplary embodiment 19, wherein the at least one agent is daratumumab.

21. The method of exemplary embodiment 18, wherein the at least one gene is LAMPS.

22. The method of exemplary embodiment 21, wherein the at least one agent is pinometostat.

23. The method of exemplary embodiment 18, wherein the at least one gene is PLACE.

24. The method of exemplary embodiment 23, wherein the at least one agent is a PI3K inhibitor.

25. The method of exemplary embodiment 24, wherein the PI3K inhibitor is selected from copanlisib, duvelisib, alpelisib, idelalisib, puquitinib, leniolisib, buparlisib, RTB101, umbralisib, TG-100-115, nemiralisib, GSK2636771, fimepinostat, tenalisib, serabelisib, INCB50465, SF1126, GDC-0077, AZD8186, ME401, IPI-549, MEN 1611 and ASN003.

26. The method of exemplary embodiment 18, wherein the at least one gene is NCAM1 (CD56).

27. The method of exemplary embodiment 26, wherein that least one agent is lorvotuzumab or mertansine.

28. The method of exemplary embodiment 18, wherein the at least one gene is SLC44A1 (CD92).

29. The method of exemplary embodiment 18, wherein the at least one gene is CD70.

30. The method of exemplary embodiment 28 or exemplary embodiment 30, wherein the at least one agent is an antibody or a CAR-T cell.

31. The method of any one of exemplary embodiments 15-30, wherein the treatment further comprises the administration of at least one therapeutically effective amount of venetoclax, azacitidine or a combination of venetoclax and azacitidine.

32. The method of any one of the preceding exemplary embodiments, wherein determining the expression level comprises PCR, high-throughput sequencing, next generation sequencing, RNA-sequencing, Northern Blot, reverse transcription PCR (RT-PCR), real-time PCR (qPCR), quantitative PCR, qRT-PCR, flow cytometry, mass spectrometry, microarray analysis, digital droplet PCR, Western Blot or any combination thereof.

33. The method of exemplary embodiment 32, wherein the RNA-sequencing is Cellular Indexing of Transcriptomes and Epitopes by Sequencing (CITE-Seq).

34. The method of any one of the preceding exemplary embodiments, wherein the biological sample is blood, a bone marrow biopsy, a bone marrow aspirate, a biopsy of a chloroma, a tissue biopsy, cerebrospinal fluid or any combination thereof.

### EXAMPLES

### Example 1

In the following non-limiting example, blood or bone marrow samples were collected from AML, patients that had been treated with a combination of venetoclax and azacitidine (Ven/aza). The patients were stratified into one of two groups. The first group were patients who showed a poor clinical response to treatment with Ven/Aza and failed to achieve a complete remission (CR) within 30 days of initiating Ven/aza treatment. Samples from this group are hereafter referred to as “Ven/aza^{R} AML samples” for Ven/aza resistant AML samples. The other group of patients were those who showed a good response to treatment with Ven/aza and achieved a CR within 30 days of initiating Ven/aza treatment. Samples from this group are hereafter referred to as “Ven/aza^{S} AML samples” for Ven/aza sensitive AML samples.

The Ven/aza^{R} AML samples and the Ven/aza^{S} AML samples were analyzed using Cellular Indexing of Transcriptomes and Epitopes by Sequencing (CITE-Seq) to measure the expression of approximately 3,000 different genes within individual cells in each patient sample. Approximately 2 to 10,000 cells per sample were analyzed using CITE-Seq, thereby generating approximately 6,000,000 to 30,000,000 data elements per each sample.

The data was then used to develop a machine learning (ML) classifier to predict Ven/aza resistant in a patient. To develop the ML classifier, the data was first down-sampled to a maximum of 300 leukemia cells per patient sample to avoid numerous samples from driving the classifier by themselves. 75% of the analyzed cells were then assigned to a training set to train the ML classifier, and 25% of the analyzed cells were then assigned to a validation set in order to validate the trained ML classifier.

Using the training set, a random forest model was trained and tuned using the 50 most variable genes in the dataset, defined using a variance stabilizing transformation, as features (see Table 2).

TABLE 2

| Gene Name | blast_importance |
|---|---|
| PCDH9 | 166.39381203009800 |
| LAMP5 | 131.87794616240100 |
| PPP1R27 | 129.64293356928200 |
| MPO | 106.35629633677500 |
| CTSG | 88.69483630003790 |
| RNASE1 | 83.24651266263390 |
| AREG | 79.0474731128395 |
| VCAN | 73.25645394470410 |
| S100A9 | 65.5110729401097 |
| S100A8 | 55.05670576135930 |
| MT2A | 54.679415678673900 |
| ELANE | 45.873172579682200 |
| RNASE3 | 44.22159775919220 |
| RETN | 43.86270818666240 |
| RND3 | 42.964105515399700 |
| FCER1A | 40.8042182627396 |
| AGR2 | 40.05173044455650 |
| FN1 | 38.53871954151630 |
| MKI67 | 38.258343734480200 |
| TPSB2 | 35.119972645913500 |
| U2AF1 | 33.567034989619900 |
| FAM83A | 31.81469455788340 |
| IFIT2 | 31.5159830997658 |
| PLBD1 | 30.580096241542300 |
| S100A12 | 29.389831833644800 |
| PRTN3 | 28.163714167273800 |
| DLK1 | 27.641535041834600 |
| MT1G | 24.79196974895410 |
| THBS1 | 23.23103123572850 |
| G0S2 | 23.142390606593000 |
| TPSAB1 | 22.254317910411700 |
| LINC00861 | 21.75890118838580 |
| HPGD | 18.017805310893300 |
| C1QA | 17.989216742739100 |
| HMOX1 | 15.445132055379800 |
| CCL4 | 14.217734157727800 |
| SERPINB2 | 11.809432250418100 |
| CCL4L2 | 11.473934854139500 |
| MS4A2 | 11.388676237358100 |
| DDIT4L | 9.069887145555960 |
| MT1H | 7.305851954245150 |
| FCGR3A | 6.334597301930770 |
| C1QB | 5.100878785915690 |
| CLC | 4.136948106642500 |
| MMP9 | 3.76376205689337 |
| PRG2 | 3.31176304150086 |
| HDC | 2.905338485720790 |
| C1QC | 2.7870557620230200 |
| CCL2 | 1.482915226431050 |
| CCL7 | 1.1682439368335000 |

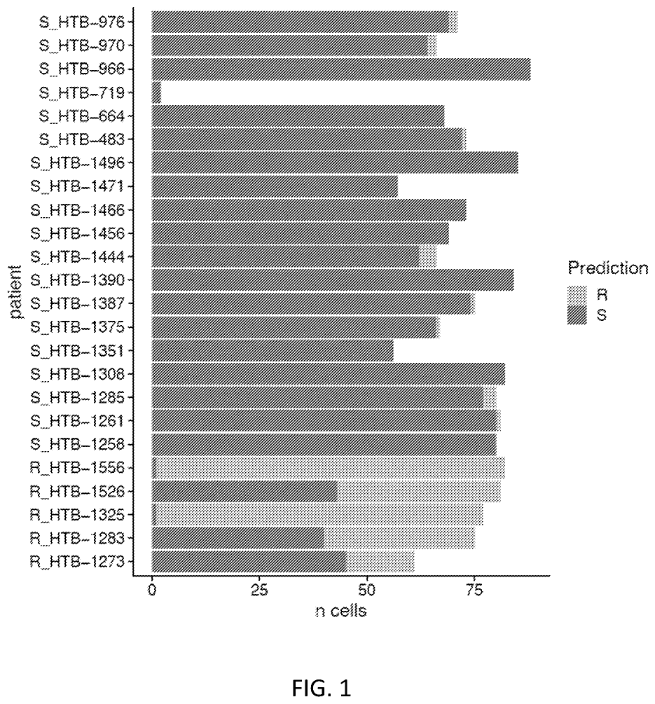

The random forest model was then tested using the expression data from the validation sample set. For each validation sample, the random forest model was used to classify the individual cells within each validation sample as either Ven/aza resistant or Ven/aza sensitive. The results of these classifications are shown in FIG. 1. The Y axis of FIG. 1 denotes whether the validation sample was from a patient who responded to treatment with Ven/aza (samples starting with “S_HTB”) or from a patient who did not respond to treatment with Ven/aza (samples starting with “R_HTB”). In the bar graphs for each sample, the number of individual cells predicted to be resistant to Ven/aza and the number of individual cells predicted to be sensitive to Ven/aza are shown. The predictions of the validation set had an area under the roc curve (AUC) of approximately 0.94.

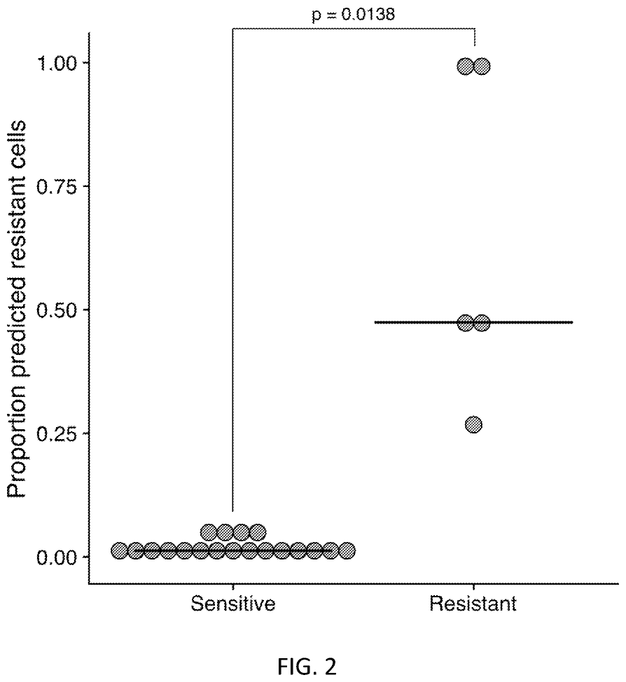

FIG. 2 shows the percentage of individual cells in each of the Ven/aza^{R} AML, samples and Ven/aza^{S} AML samples that are predicted by the random forest model resistant to Ven/aza treatment. As shown in FIG. 2, at least 25% of the individual cells in the Ven/aza^{R} AML, samples were predicted to be resistant to Ven/aza treatment.

Without wishing to be bound by theory, the results of this example show that the machine learning classifier can be used to determine the percentage of cells within a patient sample that are predicted to be resistant to treatment with Ven/aza, and that percentage can be used to further predict whether the patient will respond to Ven/aza treatment and achieve complete remission.

### Example 2

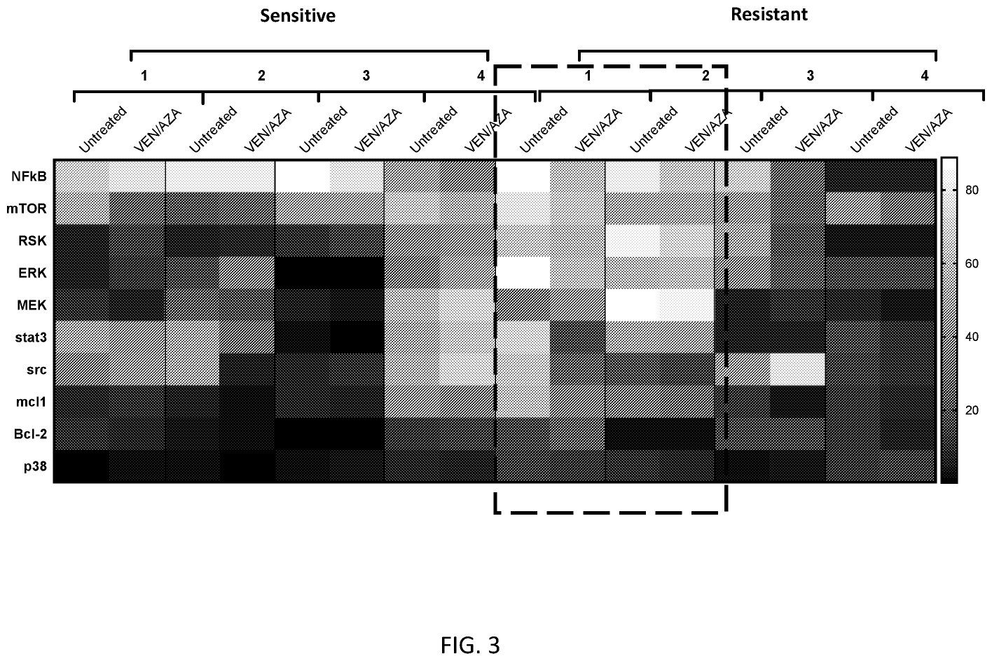

In the following non-limiting example, AML samples were obtained at the time of diagnosis for a group of patients. The patients with subsequently treated with a combination of venetoclax and azacitidine (Ven/aza). Based on each patient's response to the Ven/aza treatment, the corresponding sample was classified as either Ven/aza sensitive or Ven/aza resistant. Each patient sample was then tested by treating the sample in vitro with a combination of venetoclax and azacitidine. For comparison, control experiments were carried out where the samples were left untreated in vitro. Intracellular phosphoflow analysis was then performed. The results of the intracellular phosphoflow analysis are shown in FIG. 3. FIG. 3 shows a heat map of the phosphoflow staining intensity for a variety of different proteins in untreated and Ven/aza treated samples, both from patients who responded to Ven/aza treatment, and patients who were resistant to Ven/aza treatment. FIG. 3 shows that in a subset of AML samples in the Ven/aza resistant group (dashed box) demonstrated activation of multiple members of the PI3K/AKT/mTOR pathway. Without wishing to be bound by theory, these results demonstrate that combining Ven/aza treatment with inhibitors of the PI3K/AKT/mTOR pathway may overcome Ven/aza treatment resistance.

### Example 3

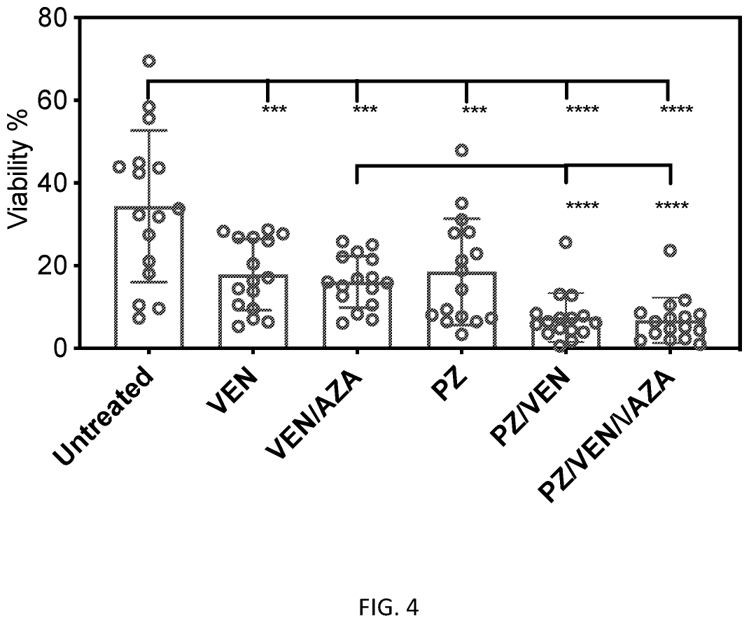

In the following non-limiting example, 16 different AML samples were treated with either venetoclax (VEN), a combination of venetoclax and azacitidine (VEN/AZA), the dual PI3K/mTOR inhibitor PF-04979064 (PZ), a combination of PF-04979064 and venetoclax (PZ/VEN), and a combination of PF-04979064, venetoclax and azacitidine (PZ/VEN/AZA). The viability of the cells after treatment were measured, and the results are shown in FIG. 4. As shown in FIG. 4, treatment with PZ/VEN or PZ/VEN/AZA was superior to treatment with VEN/AZA, VEN or PZ. Without wishing to be bound by theory, these results indicate that resistance to Ven/aza treatment may be overcome with a treatment comprising a combination of a BCL-2 inhibitor such as venetoclax and a PI3K/AKT/mTOR pathway inhibitor.

### Example 4

In the following non-limiting example, blood or bone marrow samples were collected from AML, patients that had been treated with a combination of venetoclax and azacitidine (Ven/aza). The patients were stratified into one of two groups. The first group were patients who showed a poor clinical response to treatment with Ven/Aza and failed to achieve a complete remission (CR) within 30 days of initiating Ven/aza treatment. Samples from this group are hereafter referred to as “Ven/aza^{R} AML samples” for Ven/aza resistant AML samples. The other group of patients were those who showed a good response to treatment with Ven/aza and achieved a CR within 30 days of initiating Ven/aza treatment. Samples from this group are hereafter referred to as “Ven/aza^{S} AML samples” for Ven/aza sensitive AML samples.

The Ven/aza^{R} AML samples and the Ven/aza^{S} AML samples were analyzed using Cellular Indexing of Transcriptomes and Epitopes by Sequencing (CITE-Seq) to measure the expression of approximately 3,000 different genes within individual cells in each patient sample. Approximately 2 to 10,000 cells per sample were analyzed using CITE-Seq, thereby generating approximately 6,000,000 to 30,000,000 data elements per each sample.

The data was then used to develop a machine learning (ML) classifier to predict Ven/aza resistant in a patient. To develop the ML classifier, the data was first down-sampled to a maximum of 300 leukemia cells per patient sample to avoid numerous samples from driving the classifier by themselves. 75% of the analyzed cells were then assigned to a training set to train the ML classifier, and 25% of the analyzed cells were then assigned to a validation set in order to validate the trained ML classifier.

Using the training set, a random forest model was trained and tuned using the 50 most variable genes in the dataset, defined using a variance stabilizing transformation, as features (see Table 3).

TABLE 3

| Gene Name | blast_importance |
|---|---|
| MT2A | 241.98 |
| MPO | 209.82 |
| DLK1 | 196.94 |
| AZU1 | 177.11 |
| S100A9 | 168.26 |
| PPP1R27 | 164.76 |
| ELANE | 140.68 |
| S100A8 | 140.30 |
| CTSG | 138.34 |
| AVP | 131.04 |
| RNASE1 | 126.70 |
| TPSB2 | 110.71 |
| TPSAB1 | 107.72 |
| POU4F1 | 100.60 |
| DNTT | 91.65 |
| FCER1A | 83.23 |
| CENPF | 81.50 |
| PRTN3 | 78.01 |
| S100A12 | 74.80 |
| IFIT3 | 68.70 |
| THBS1 | 66.65 |
| G0S2 | 65.02 |
| IFIT2 | 58.13 |
| MKI67 | 56.44 |
| HPGD | 53.23 |
| TOP2A | 48.43 |
| C1QA | 48.38 |
| HBD | 42.57 |
| GNLY | 42.42 |
| MT1G | 41.47 |
| CTSL | 40.83 |
| CCL4 | 39.84 |
| FN1 | 36.14 |
| SERPINB2 | 35.11 |
| C1QB | 31.34 |
| PRG2 | 29.67 |
| FCGR3A | 23.23 |
| DEFB1 | 22.06 |
| DEFA4 | 21.66 |
| UBE2C | 21.10 |
| PRSS2 | 18.56 |
| PPBP | 15.78 |
| CLC | 14.43 |
| IFI27 | 12.80 |
| DEFA3 | 12.46 |
| MS4A2 | 11.82 |
| CCL2 | 8.14 |
| LTF | 4.28 |
| TCN1 | 3.19 |
| CCL7 | 2.82 |

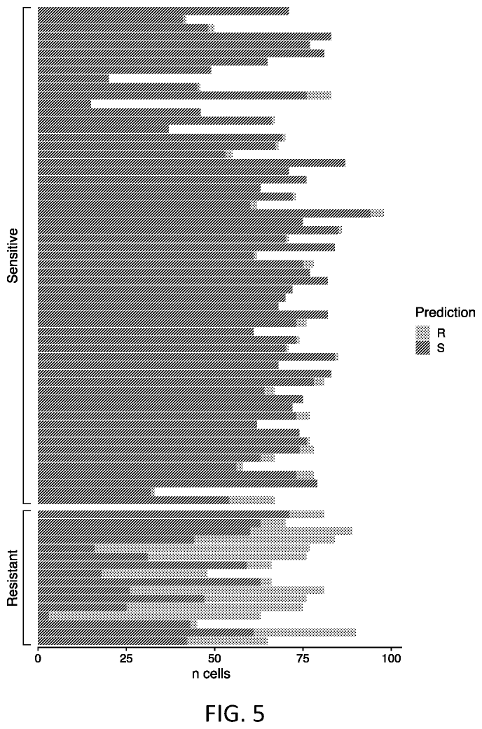

The random forest model was then tested using the expression data from the validation sample set. For each validation sample, the random forest model was used to classify the individual cells within each validation sample as either Ven/aza resistant or Ven/aza sensitive. The results of these classifications are shown in FIG. 5. The Y axis of FIG. 5 denotes whether the validation sample was from a patient who responded to treatment with Ven/aza (“Sensitive”) or from a patient who did not respond to treatment with Ven/aza (“Resistant”). In the bar graphs for each sample, the number of individual cells predicted to be resistant to Ven/aza and the number of individual cells predicted to be sensitive to Ven/aza are shown. The predictions of the validation set had an area under the roc curve (AUC) of approximately 0.89.

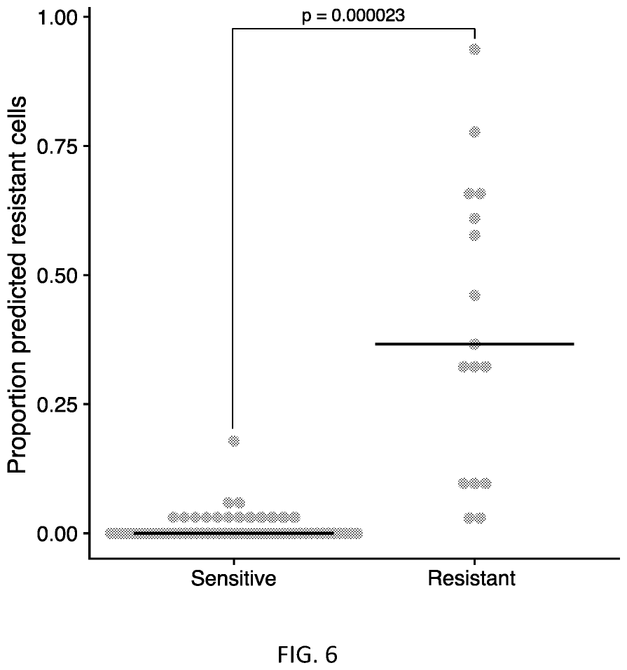

FIG. 6 shows the percentage of individual cells in each of the Ven/aza^{R} AML, samples and Ven/aza^{S} AML samples that are predicted by the random forest model resistant to Ven/aza treatment. As shown in FIG. 6, at least 25% of the individual cells in 11 of the 16 Ven/aza^{R} AML samples were predicted to be resistant to Ven/aza treatment.

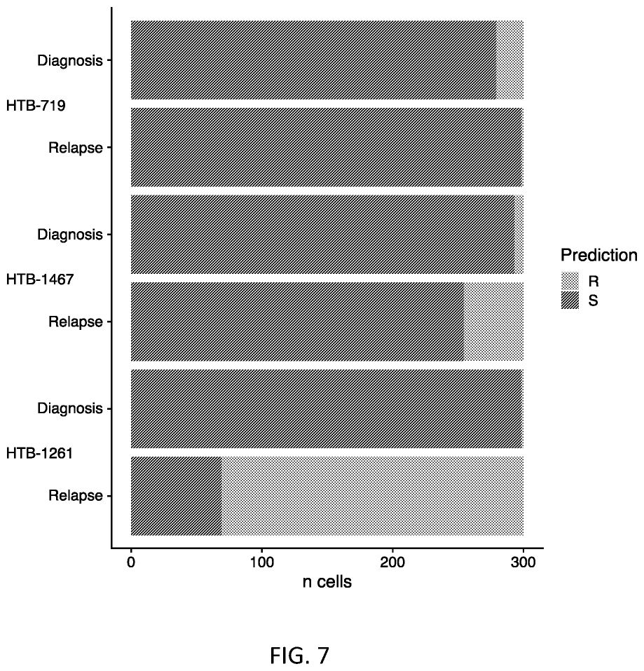

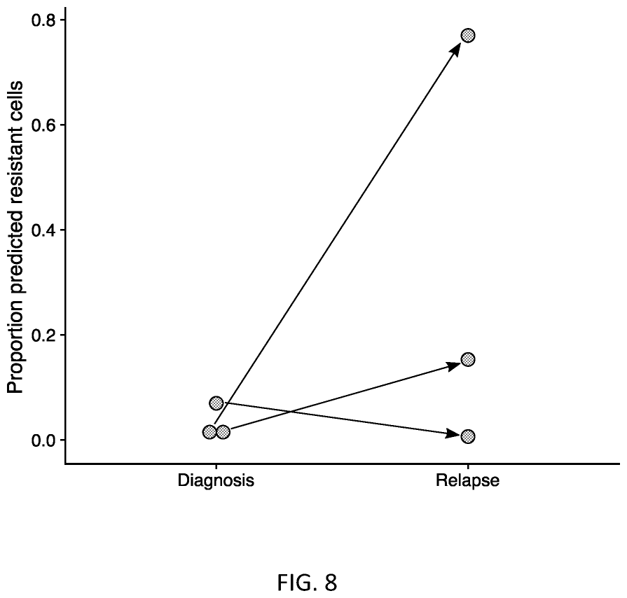

The random forest model was also used to classify the individual cells within three pairs of diagnosis and relapse samples from patients initially sensitive to Ven/aza. The results of these classifications are shown in FIG. 7 and FIG. 8. As shown in FIG. 7 and FIG. 8, while the majority of cells are predicted to be sensitive to Ven/aza at diagnosis, two of the three patients demonstrate an increased proportion of cells predicted to be resistant to Ven/aza at relapse.

Without wishing to be bound by theory, the results of this example show that the machine learning classifier can be used to determine the percentage of cells within a patient sample that are predicted to be resistant to treatment with Ven/aza, and that percentage can be used to further predict whether the patient will respond to Ven/aza treatment and achieve complete remission.

What is claimed is:

1. A method of identifying a subject having acute myeloid leukemia (AML) that will be resistant to treatment with a combination of venetoclax and azacitidine, the method comprising:
a) measuring the expression levels of at least 10 genes in a plurality of leukemia cells isolated from a biological sample from the subject,
wherein the at least 10 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C;
b) classifying a measured cell as resistant to treatment with a combination of venetoclax and azacitidine or classifying a measured cell as responsive to treatment with a combination of venetoclax and azacitidine based on the expression levels measured in step (a);
c) determining the percentage of leukemia cells in the plurality of leukemia cells that are classified as resistant to treatment with a combination of venetoclax and azacitidine;
d) comparing the percentage from step (c) to a predetermined cutoff percentage; and
e) identifying that the subject will be resistant to treatment with a combination of venetoclax and azacitidine when the percentage from step (c) is greater than the predetermined cutoff percentage.

2. The method of claim 1, wherein classifying a measured cell as resistant to treatment with a combination of venetoclax and azacitidine or classifying a measured cell as responsive to treatment with a combination of venetoclax and azacitidine based on the expression levels measured in step (a) comprises:
i) determining a score based on the expression level of the at least 10 genes, wherein the score is determined using a machine learning classifier;
ii) comparing the score determined in step (i) to a predetermined cutoff value; and
iii) classifying the cell as resistant to treatment with a combination of venetoclax and azacitidine when the score is greater than or equal to the predetermined cutoff value or classifying the cell as responsive to treatment with a combination of venetoclax and azacitidine when the score is less than the predetermined cutoff value.

3. The method of claim 1, wherein classifying a measured cell as resistant to treatment with a combination of venetoclax and azacitidine or classifying a measured cell as responsive to treatment with a combination of venetoclax and azacitidine based on the expression levels measured in step (a) comprises:
i) determining a score based on the expression level of the at least 10 genes,
wherein the score is determined using a machine learning classifier;
ii) comparing the score determined in step (i) to a predetermined cutoff value; and
iii) classifying the cell as resistant to treatment with a combination of venetoclax and azacitidine when the score is less than or equal to the predetermined cutoff value or classifying the cell as responsive to treatment with a combination of venetoclax and azacitidine when the score is greater than the predetermined cutoff value.

4. The method of claim 2 or claim 3, wherein the machine learning classifier is trained and validated using the expression levels of the at least 10 genes measured in at least two training samples,
wherein at least one of the at least two training samples comprises leukemia cells isolated from a subject that is responsive to treatment with venetoclax and azacitidine, and
wherein at least one of the at least two training samples comprises leukemia cells isolated from a subject that is resistant to treatment with venetoclax and azacitidine.

5. The method of any one of the preceding claims, wherein step (a) comprises measuring the expression levels of at least 25 genes in the plurality of leukemia cells, wherein the at least 25 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C.

6. The method of any one of the preceding claims, wherein step (a) comprises measuring the expression levels of at least 40 genes in the plurality of leukemia cells, wherein the at least 40 genes are selected from AVP, AZU1, C1QA, C1QB, CCL2, CCL4, CCL7, CENPF, CLC, CTSG, CTSL, DEFA3, DEFA4, DEFB1, DLK1, DNTT, ELANE, FCER1A, FCGR3A, FN1, GOS2, GNLY, HBD, HPGD, IFI27, IFIT2, IFIT3, LTF, MKI67, MPO, MS4A2, MT1G, MT2A, POU4F1, PPBP, PPP1R27, PRG2, PRSS2, PRTN3, RNASE1, S100A8, S100A9, S100A12, SERPINB2, TCN1, THBS1, TOP2A, TPSAB1, TPSB2, and UBE2C.

7. The method of any of the preceding claims, wherein the plurality of leukemia cells comprises at least about 300 leukemia cells.

8. The method of any one of the preceding claims, wherein the leukemia cells comprise acute myeloid leukemia blast cells.

9. The method of any one of the preceding claims, wherein the leukemia cells comprise leukemia stem cells.

10. The method of claim 9, wherein the leukemia stem cells comprise reactive oxygen species-low leukemia stem cells.

11. The method of any one of the preceding claims, wherein the predetermined cutoff percentage is at least about 25%.

12. The method of any of the preceding claims, further comprising providing a treatment recommendation to the subject that is identified as a subject that is resistant to treatment with a combination of venetoclax and azacitidine, wherein the treatment recommendation comprises recommending the administration of at least one therapeutically effective amount of at least one alternative therapy.

13. The method of any of the preceding claims, further comprising administering to the subject identified as resistant to treatment with a combination of venetoclax and azacitidine at least one therapeutically effective amount of at least one alternative therapy.

14. The method of claim 12 or claim 13, wherein the at least one alternative therapy comprises anti-cancer therapy, chemotherapy, targeted drug therapy, radiation therapy, immunotherapy, stem cell transplant or any combination thereof.

15. A method of providing an AML treatment recommendation for a subject, the method comprising:
a) determining the expression level of at least one gene in a plurality of leukemia stem cells isolated from a biological sample from the subject, wherein the at least one gene is selected from NFκB, mTOR, RSK, ERK, MEK, stat3, src, mcl1;
b) comparing the expression level of the at least one gene in the measured cells to a corresponding predetermined cutoff value;
c) determining the percentage of leukemia cells in the plurality of leukemia cells that exhibit an expression level of the at least one gene that is greater than the corresponding predetermined cutoff value;
d) comparing the percentage from step (c) to a predetermined cutoff percentage; and
e) recommending a treatment comprising the administration of at least one therapeutically effective amount of at least one agent that targets the PI3K/AKT/mTOR pathway when the percentage from step (c) is greater than the predetermined cutoff percentage.

16. The method of claim 15, wherein the at least one agent that targets the PI3K/AKT/mTOR is an agent that inhibits at least one of PI3K, AKT and mTOR.

17. The method of claim 15, wherein the at least one agent that targets the PI3K/AKT/mTOR pathway is selected from everolimus, temsirolimus, sirolimus, CC-223, vistusertib, nab-rapamycin, CC-115, sapanisertib, copanlisib, duvelisib, alpelisib, idelalisib, puquitinib, leniolisib, buparlisib, RTB101, umbralisib, TG-100-115, nemiralisib, GSK2636771, fimepinostat, tenalisib, serabelisib, INCB50465, SF1126, GDC-0077, AZD8186, ME401, IPI-549, MEN 1611, ASN003, bimiralisib, GDC0084, voxtalisib, LY3023414, gedatolisib, ARG-092, MK-2206, iapatasertib, uprosertib, capivasertib, triciribine, ARQ-751, PF-04979064 and PF-04691502.

18. A method of providing an AML treatment recommendation for a subject, the method comprising:
a) determining the expression level of at least one gene in a plurality of leukemia stem cells isolated from a biological sample from the subject, wherein the at least one gene is selected from CD38, LAMPS, SLC44A1 (CD92), PLAC8, NCAM1 (CD56) and CD70;
b) comparing the expression level of the at least one gene in the measured cells to a corresponding predetermined cutoff value;
c) determining the percentage of leukemia cells in the plurality of leukemia cells that exhibit an expression level of the at least one gene that is greater than the corresponding predetermined cutoff value;
d) comparing the percentage from step (c) to a predetermined cutoff percentage;
e) recommending a treatment comprising the administration of at least one therapeutically effective amount of at least one agent that targets the at least one gene when the percentage from step (c) is greater than the predetermined cutoff percentage.

19. The method of claim 18, wherein the at least one gene is CD38.

20. The method of claim 19, wherein the at least one agent is daratumumab.

21. The method of claim 18, wherein the at least one gene is LAMPS.

22. The method of claim 21, wherein the at least one agent is pinometostat.

23. The method of claim 18, wherein the at least one gene is PLAC8.

24. The method of claim 23, wherein the at least one agent is a PI3K inhibitor.

25. The method of claim 24, wherein the PI3K inhibitor is selected from copanlisib, duvelisib, alpelisib, idelalisib, puquitinib, leniolisib, buparlisib, RTB101, umbralisib, TG-100-115, nemiralisib, GSK2636771, fimepinostat, tenalisib, serabelisib, INCB50465, SF1126, GDC-0077, AZD8186, ME401, IPI-549, MEN 1611 and ASN003.

26. The method of claim 18, wherein the at least one gene is NCAM1 (CD56).

27. The method of claim 26, wherein that least one agent is lorvotuzumab or mertansine.

28. The method of claim 18, wherein the at least one gene is SLC44A1 (CD92).

29. The method of claim 18, wherein the at least one gene is CD70.

30. The method of claim 28 or claim 30, wherein the at least one agent is an antibody or a CAR-T cell.

31. The method of any one of claims 15-30, wherein the treatment further comprises the administration of at least one therapeutically effective amount of venetoclax, azacitidine or a combination of venetoclax and azacitidine.

32. The method of any one of the preceding claims, wherein determining the expression level comprises PCR, high-throughput sequencing, next generation sequencing, RNA-sequencing, Northern Blot, reverse transcription PCR (RT-PCR), real-time PCR (qPCR), quantitative PCR, qRT-PCR, flow cytometry, mass spectrometry, microarray analysis, digital droplet PCR, Western Blot or any combination thereof.

33. The method of claim 32, wherein the RNA-sequencing is Cellular Indexing of Transcriptomes and Epitopes by Sequencing (CITE-Seq).

34. The method of any one of the preceding claims, wherein the biological sample is blood, a bone marrow biopsy, a bone marrow aspirate, a biopsy of a chloroma, a tissue biopsy, cerebrospinal fluid or any combination thereof.
